(19) United States
(12) Patent Application Publication
(10) Pub. No.: US 2018/0221463 A1
(43) Pub. Date: Aug. 9, 2018

(54) Modified NK Cells and Uses Thereof

(71) Applicant: The Chinese University of Hong Kong, Shatin (CN)

(72) Inventor: Hui Yao LAN, Shatin (CN)

(21) Appl. No.: 15/863,159

(22) Filed: Jan. 5, 2018

Related U.S. Application Data
(60) Provisional application No. 62/446,106, filed on Jan. 13, 2017.

(51) Int. Cl. A61K 39/00; C12N 5/0783; A61K 9/00; A61P 35/00; A61K 45/06

(52) U.S. Cl. CPC A61K 39/0011; C12N 5/0646; A61K 2039/515; A61P 35/00; A61K 45/06; A61K 9/0019

(57) ABSTRACT

The present invention provides for modified natural killer (NK) cells that are resistant to TGF-β stimulation due to suppressed Smad3 activity in these cells. Also provided are compositions comprising these modified NK cells, as well as methods of treating cancer by using the cells.

### RELATED APPLICATIONS

This application claims priority to U.S. Provisional Patent Application No. 62/446,106, filed Jan. 13, 2017, the contents of which are herein incorporated in the entirety for all purposes.

### BACKGROUND OF THE INVENTION

Cancer is a generic term for a large group of diseases that can affect any part of the body. One defining feature of cancer is the rapid proliferation of abnormal cells that grow beyond their usual boundaries. Cancer cells can then invade adjoining parts of the body and spread to other organs in a process known as metastasis. Metastasis is the main cause of death from cancer and can also be promoted by the cells surrounding the cancer called cancer stromal cells or cancer microenviroments.

According to the World Health Organization (WHO), cancer is a leading cause of death worldwide, accounting for 7.6 million deaths (around 13% of all deaths) in 2008. Lung, stomach, liver, colon and breast cancer cause the most cancer deaths each year. Despite intense research effort and technological advancement in biomedical sciences, deaths from cancer worldwide are projected to continue rising, with an estimated 13.1 million deaths in 2030.

Because of the prevalence of cancer and its significant impact on humanity, there remains an urgent need to develop new and more effective strategies for cancer treatment. The present invention addresses this and other related needs in that it provides a new strategy in anti-cancer treatment: by genetically modifying natural killer (NK) cells such that Smad3 expression and/or activity is substantially suppressed in these cells, the cancer-killing activities of these NK cells are notably enhanced. These Smad3-knockdown NK cells are therefore novel and effective tools in cancer therapy.

### BRIEF SUMMARY OF THE INVENTION

The invention relates to novel methods and compositions useful for cancer immunotherapy. In particular, the present inventor discovered that, by inhibiting the endogenous expression and activity of Smad3 in an immune effector cell such as a natural kill (NK) cell, the effector cell becomes resistant to TGF-β stimulation and exhibits significantly enhanced cancer-killing activities. Thus, in the first aspect, the present invention provides a novel, modified immune effector cell (e.g., an NK cell), where the Smad3 activity in the modified cell is inhibited or reduced/suppressed, or even completely eliminated, compared to a unmodified parent cell of the same kind. For example, in the modified cell, Smad3 activity is inhibited by 50%, 70%, 80% or more compared to the unmodified parent cell. In some cases, Smad3 activity is abolished. The change in Smad3 activity may be due to the Smad3 genomic sequence having been altered, such as by way of substitution, deletion, insertion, or complete removal of the entire sequence. In some cases, the modified cell comprises in its genome an exogenous sequence encoding a polynucleotide sequence that corresponds to or is complementary to at least a segment of the Smad3 genomic sequence, the expression of such sequence (especially at RNA level) then leads to the suppression of Smad3 activity. For example, inhibition/elimination of Smad3 activity may be achieved by way of a virus and/or non-virus vector encoding Smad3-disrupting sequence such as shRNA, anti-sense, CRISPR-Cas9, or a specific inhibitor for Smad3. In some cases, the effector cell is an NK cell, for example, the parent NK cell is human NK92 cell. In the alternative, other cell types may be used to produce the modified Smad3 knock-down cells, for instance, B cell subsets, T cell subsets, macrophages, dendritic cells, neutrophils, eosinophils, and mast cells, as well as non-immune cells including stem cells or fibroblasts. Suitable cells for such manipulation may be naturally occurring cells as well as artificially engineered or recombinantly produced cells, for example, genetically modified cells such as chimeric antigen receptor (CAR) T cells.

In a second aspect, the present invention provides composition comprising the modified cell, especially a modified NK cell, described above and herein and a physiologically acceptable excipient. In some embodiments, the composition is formulated for injection, for example, it may be formulated for subcutaneous, intramuscular, intravenous, intraperitoneal, or intratumoral injection. In some embodiments, the composition is formulated in a dosage form for administration to a patient, for example, it may be formulated and packaged in multiple units (e.g., vials), each having adequate amount for each administration for a multiple-day or multiple-week treatment course.

In a third aspect, the present invention provides a method for treating cancer. The method includes the step of administration to a cancer patient an effective number of the modified cell, especially NK cell described above and herein, to a patient in need thereof. In some embodiments, the administration step comprises injection, for example, subcutaneous, intramuscular, intravenous, intraperitoneal, or intratumoral injection. In some embodiments, the administration is performed daily, once every two days, weekly, once every two weeks, or monthly. In some embodiments, the method also includes co-administration of a second anti-cancer therapeutic agent to the patient. In some embodiments, the cancer is one induced by bacterial or virus infection, toxins, drugs, genetics, smoking, irradiation, and chemicals. The cancer to be treated may be primary cancer or metastatic cancers, for example, various types of skin cancer (e.g., melanoma), liver cancer, lung carcinoma, gastric and colon cancers, various types of leukemia, T and B cells lymphoma, sarcoma, and other types of cancer in the digestive, reproduction, and nervous systems.

In a related aspect, the present invention provides a practical application of the modified immune effector cells: they can be used for the manufacture of a medicament for the treatment of cancer in accordance with the method describe above and herein.

### BRIEF DESCRIPTION OF THE DRAWINGS

FIGS. 1A-1D: Knockdown SMAD3 from NK-92 cells enhances the cancer-killing activities in vitro. FIGS. 1A and 1B, Real-time PCR and western blot analysis show that transduction of shRNA-SMAD3 significantly down-regulates Smad3 mRNA and protein expression in NK-92 cells. FIGS. 1C and 1D, Comparison of the cancer-killing activity between NK-92-S3KD and NK-92-EV cells against human hepatoma HepG2 and melanoma A375 cells in the presence or absence of TGF-β1 (5 ng/ml). The cytotoxicity was measured at various E:T ratios by cell-mediated cytotoxicity assay kit. Data represent mean±SD for groups of three independent experiments. ### P<0.001 versus NK-92-EV cells; **P<0.01, ***P<0.001 versus TGF-β1-treated cells; ^{§§§} <0.001 versus TGF-β1-treated NK-92-EV cells.

FIGS. 2A-2F: Disruption of SMAD3 enhances production of anti-cancer cytokines in NK-92-S3KD cells. FIGS. 2A-2C, Real-time PCR. D-F, ELISA. Results show that knockdown of Smad3 from the NK-92 cells protects against TGF-β1 (5 ng/ml) induced suppressive effect on both mRNA and protein expression of IFN-γ, granzyme B and perforin. Data represent mean±SD for groups of three independent experiments. *P<0.05, **P<0.01, ***P<0.001 versus TGF-β1 0 ng/ml; ## P<0.01, ### P<0.001 versus NK-92-EV cells.

FIGS. 3A-3C: Silencing Smad3 enhanced production of GM-CSF by HK92-S3KD cells in vitro. (FIG. 3A) cytokine array analysis; (FIG. 3B) Real-time RT-PCR; (FIG. 3C) ELISA. Results show that addition of TGF-β1 suppressed GM-CSF expression in both mRNA and protein levels, which was prevented by silencing Smad3 in HK92-S3KD cells. Data shown are representative of 3 independent experiments. ***P<0.001, versus TGF-β1 0 ng/ml; ### P<0.001, versus NK-92-EV

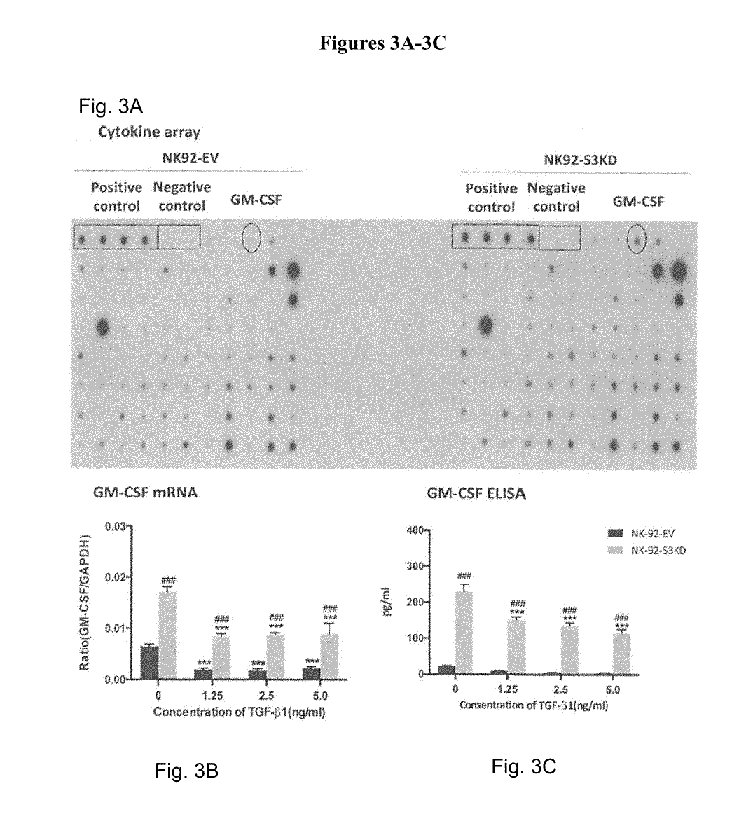

FIGS. 4A-4G: Disrupted Smad3 largely enhances NK cell mediated anti-tumor effect on HepG2 and A375-bearing NOD/SCID mice. FIG. 4A, Tumor volume of HepG2. FIG. 4B, Luciferase intensity imaging of HepG2-Luc tumor bearing mice. FIG. 4C, Tumor weight of HepG2. FIG. 4D, Tumor size of HepG2. FIG. 4E, Tumor volume of A375. FIG. 4F, Tumor weight of A375. FIG. 4G, Tumor size of A375. Data represent mean±SD for groups of 5-7 mice. **P<0.01, ***P<0.001 versus saline group; #P<0.05, ### P<0.001 versus NK-92-EV-treated group.

FIGS. 5A-5F: Immunotherapy of NK-92-S3KD cells systemically increases anticancer cytokines in HepG2-bearing mice. FIGS. 5A-5C, Tumor tissue-derived human IFN-γ, granzyme B and perforin. FIGS. 5D-5F, Serum levels of human IFN-γ, granzyme B and perforin. Results show that the levels of IFN-γ, granzyme B and perforin in both tumor tissues and serum of HepG2-bearing mice on day 28 after tumor inoculation are doubled in the tumor-bearing mice treated with NK-92-S3KD cells compared with parental cells. Data represent mean±SD for groups of at least 6 mice. **P<0.01, ***P<0.001 versus Saline group; ### P<0.001 versus NK-92-EV group.

FIGS. 6A-6F: Regulatory mechanism of IFN-γ production by the SMAD3-dependent E4BP4 pathway in NK-92 cells in vitro. FIG. 6A, Western blot analysis of Smad3 phosphorylation in NK-92. FIG. 6B, Real-time PCR of E4BP4 mRNA expression in NK-92-EV cells. FIG. 6C, Western blot analysis of E4BP4 protein expression in NK-92 cells treated with 5 ng/ml TGF-β1. FIGS. 6D-6F, Real-time PCR, western blot analysis of E4BP4 and ELISA of IFN-γ in NK-92-EV cells treated with SIS3 (5 μM). Data represent mean±SD for three independent experiments. *P<0.05, **P<0.01, ***P<0.001 versus 0 ng/ml of TGF-β 1; ### P<0.001 versus NK-92-EV or TGF-β1-treated only.

FIGS. 7A-7F: IFNG is an E4BP4 target gene that is regulated by SMAD3 in mature human NK cells. FIG. 7A, A Smad3-binding site on the 3′ UTR of E4BP4 (NFIL3). FIG. 7B, ChIP assay detects an increased Smad3-E4BP4 binding in response to TGF-β1 (5 ng/ml) at 12 h. FIG. 7C, The promoter activity of E4BP4. Overexpression of Smad3 protein largely suppresses promoter activities of E4BP4, which is prevented when the predicted SMAD3-binding site on the 3′UTR of E4BP4 genomic sequence is deleted. FIG. 7D, An E4BP4-binding site on the promoter region of IFNG gene is predicted by ECR browser. FIG. 7E, ChIP assay detects E4BP4 binding on INFG that is greatly reduced at 12 h in response to TGF-β1 (5 ng/ml). FIG. 7F, Overexpression of E4BP4 protein enhances the promoter activity of IFNG that is significantly prevented when the predicted E4BP4-binding site on the IFNG promoter sequence is deleted. Data represent mean±SD for three independent experiments. ***P<0.001 versus IFNG-blank.

FIGS. 8A-8D: Double blockade of SMAD3 and E4BP4 blunts IFN-γ production by NK-92 cells in vitro. FIGS. 8A and 8B, Real-time PCR and western blot analysis of E4BP4 mRNA and protein expression in NK-92 cells. FIGS. 8C and 8D, Real-time PCR and ELISA of IFN-γ mRNA and protein levels in NK-92 cells. Data represent mean±SD for three independent experiments. *P<0.05, ***P<0.001 versus NK-92-EV; ### P<0.001 versus NK-92-S3KD cells.

FIGS. 9A-9C: Construction of a recombinant plasmid expressing shRNA targeting human SMAD3 mRNA. FIG. 9A, Map of plasmid backbone PLVX-ShRNA1-Puro; FIG. 9B, The restricted DNA products of recombinant plasmid digested with restriction endonuclease Xho I was separated by 1% agarose gel electrophoresis. Lane M, DNA marker; Lane 1, restricted DNA products; FIG. 9C, The result was confirmed by DNA Sequencing.

FIG. 10: Disruption of Smad3 does not influence cell proliferation of NK-92 cells in the absence or presence of TGF-β1. NK-92-EV cells or NK-92-S3KD cells were seeded in a density of 1×10⁴/well in 96-well plates with TGF-β1 for 44 hours. MTT was added and continuously incubated for 4 hours. Cell viability was determined by the absorbance at a wavelength of 490 nm. Each bar represents mean±SD for three independent experiments. *P<0.05, **P<0.01 versus TGF-β1 0 ng/ml.

FIG. 11: Disruption of Smad3 does not influence the inhibitory effect of TGF-β1 on NKG2D expression in NK-92 cells. NK-92-EV and NK-92-S3KD cells were stimulated with TGF-β1 for 3 hours and mRNA level of NKG2D was measured. Each bar represents mean±SD from three independent experiments. *P<0.05, **P<0.01, *** P<0.001 versus TGF-β1 0 ng/ml.

FIGS. 12A-12-D: NK-92-S3KD cell transfer does not cause significant adverse effects in HepG2 bearing mice. Mouse serum was collected from HepG2-Luc bearing mice 28 days after initiation of NK cell therapy. FIG. 12A, Creatinine; FIG. 12B, Lactate dehydrogenase; FIG. 12C, Alanine aminotransferase and FIG. 12D, Aspartate aminotransferase were measured with commercial kits. Each bar represents mean±SD from groups of 5-7 mice. ns means no significance.

FIGS. 13A-13C: Construction of a recombinant plasmid expressing shRNA that targets human E4BP4 mRNA. FIG. 13A, Map of plasmid backbone pLVX-ShRNA2-Neo. FIG. 13B, The restricted DNA products of recombinant plasmid digested with restriction endonuclease Miu I was separated by 1% agarose gel electrophoresis. Lane M, DNA marker; Lane1, restricted DNA products. FIG. 13C, The result was confirmed by DNA Sequencing.

FIGS. 14A-14B: Construction of human SMAD3 mRNA expressing plasmid (pcDNA3.1+SMAD3) and recombinant plasmids containing E4BP4 3′UTR (psi-CHECK2-E4BP4 3′UTR). FIG. 14A, CDS (coding sequence) region of human SMAD3 was amplified and cloned into pcDNA3.1+ vector. The restricted DNA products of recombinant plasmid digested with Xho I/Kpn I was separated by 1% agarose gel electrophoresis, which gave a band of 963 bp corresponding to the size of CDS of Smad3. Lane M, DNA marker; lane 1,2,3 restricted DNA products, example of positive clone was highlighted; FIG. 14B, 3′UTR of E4BP4 was cloned into psi-CHECK2. The inserted E4BP4 3′UTR (300 bp) was validated by restriction endonuclease digested with Not Xho I. Lane M, DNA marker; lane 1,2,3 restricted DNA products, example of positive clone was highlighted as indicated.

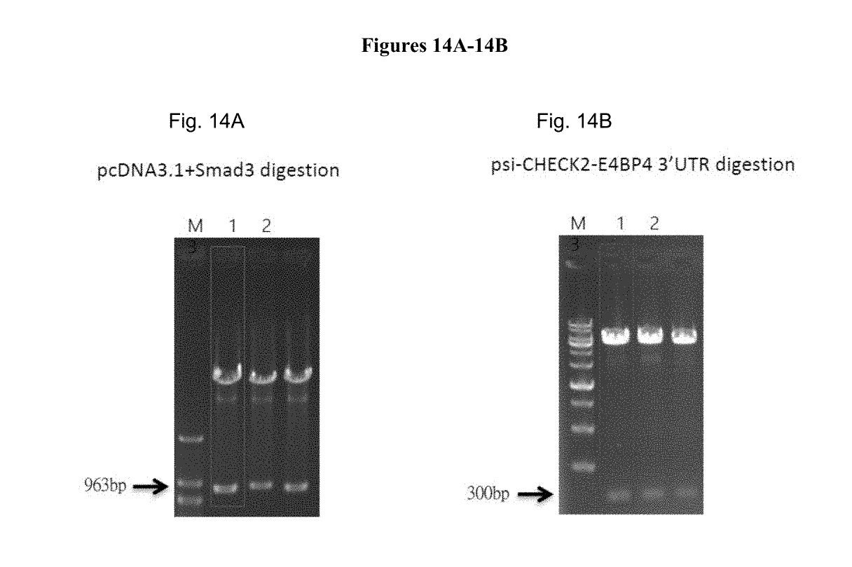

FIGS. 15A-15B: Construction of E4BP4 expression plasmid (pcDNA3.1+E4BP4) and recombinant plasmids containing pGL3-IFNG promoter (pGL3-IFNG promoter). FIG. 15A, CDS region of human E4BP4 was amplified and cloned into pcDNA3.1+ vector. The restricted DNA products of recombinant plasmid digested with Xho I/BamH I was separated by 1% agarose gel electrophoresis, which gave a band of 1389 bp corresponding to the size of CDS of E4BP4. Lane M, DNA marker; lane 1,2,3 restricted DNA products, example of positive clone was highlighted; FIG. 15B, IFNG promoter was cloned into pGL3 plasmid. The accuracy of inserted IFNG promoter (907 bp) was identified by restriction endonuclease digested with Miu I/Xho I. Lane M, DNA marker; lane 1,2,3 restricted DNA products, example of positive clone was highlighted as indicated.

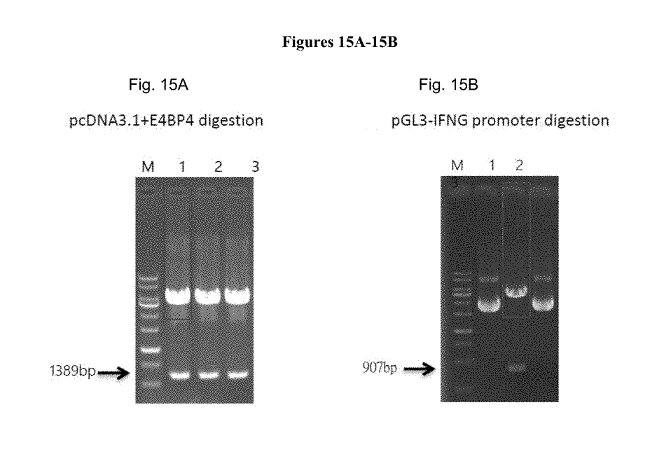

### DEFINITIONS

The term “inhibiting” or “inhibition,” as used herein, refers to any detectable negative effect on a target biological process, such as RNA/protein expression of a target gene, the biological activity of a target protein, cellular signal transduction, cell proliferation, tumorigenicity, and metastatic potential. Typically, an inhibition is reflected in a decrease of at least 10%, 20%, 30%, 40%, 50%, 60%, 70%, 80%, 90% or greater in target process (e.g., expression or activity of Smad3, Smad3-mediated signaling or cancer proliferation), or any one of the downstream parameters mentioned above, when compared to a control. “Inhibition” further includes a 100% reduction, i.e., complete elimination or abolition of a target biological process or signal. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “increasing,” “increase,” “enhancing,” or “enhancement” are used in this disclosure to encompass positive changes at different levels (e.g., at least 10%, 20%, 30%, 40%, 50%, 60%, 70%, 80%, 90%, 100%, 200%, or greater such as 3, 5, 8, 10, 20-fold increase compared to a control level) in a target process or signal.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “effective amount” or “effective number,” as used herein, refers to an amount or number that produces therapeutic effects for which a substance is administered. The effects include the prevention, correction, or inhibition of progression of the symptoms of a disease/condition and related complications to any detectable extent. The exact amount will depend on the nature of the therapeutic agent, the manner of administration, and the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter.

An “antibody” refers to a polypeptide substantially encoded by an immunoglobulin gene or immunoglobulin genes, or fragments thereof, which specifically bind and recognize an antigen, for example, the Smad3 protein. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies may exist in various forms, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology.

Further modification of antibodies by recombinant technologies is also well known in the art. For instance, chimeric antibodies combine the antigen binding regions (variable regions) of an antibody from one animal with the constant regions of an antibody from another animal. Generally, the antigen binding regions are derived from a non-human animal, while the constant regions are drawn from human antibodies. The presence of the human constant regions reduces the likelihood that the antibody will be rejected as foreign by a human recipient. On the other hand, “humanized” antibodies combine an even smaller portion of the non-human antibody with human components. Generally, a humanized antibody comprises the hypervariable regions, or complementarity determining regions (CDR), of a non-human antibody grafted onto the appropriate framework regions of a human antibody. Antigen binding sites may be wild type or modified by one or more amino acid substitutions, e.g., modified to resemble human immunoglobulin more closely. Both chimeric and humanized antibodies are made using recombinant techniques, which are well-known in the art (see, e.g., Jones et al. (1986) Nature 321:522-525).

Thus, the term “antibody,” as used herein, also includes antibody fragments either produced by the modification of whole antibodies or antibodies synthesized de novo using recombinant DNA methodologies (e.g., single chain Fv, a chimeric or humanized antibody).

The term “Smad3-knockdown,” as used herein, describes a cell that has been modified, especially genetically modified, and therefore exhibits inhibited Smad3 expression and/or activity in comparison with the unmodified parent cell. Immune effector cells, such as the NK cells, are used to generate Smad3-knockdown cells, including stable cell lines that over multiple passages (e.g., at least 5, 10, or 20 or more passages) retain the characteristic of low to no Smad3 expression or activity. Smad3, or Mothers against decapentaplegic homolog 3, is an intracellular signal transducer and transcriptional modulator activated by transforming growth factor (TGF)-β and activin type 1 receptor kinases. The amino acid sequence and corresponding polynucleotide coding sequence for Human Smad3 are provided in GenBank Accession Number AAB80960 and U68019, respectively. A Smad3-knockdown cell is one that has been modified to have endogenous Smad3 expression or activity level reduced by at least 25%, 50%, 75%, 80%, 90% or more in comparison with a control cell (a parent cell not having been modified). In some cases, the Smad3-knockdown cell has a suppressed level of Smad3 expression and/or activity but a detectable residual expression/activity remains. In other cases, the Smad3-knockdown cell will have no detectable expression or activity of Smad3. Due to the suppressed Smad3 expression and activity, a Smad3-knockdown cell is resistant or less responsive (in some cases completely non-responsive) to TGF-β signaling and is therefore also referred to as “TGF-β tolerant.” Similarly, the term “E4BP4- or IFNG-enhanced cells” refer to cells that have been modified, especially genetically modified, to possess an increase in the expression of the E4BP4 or IFNG gene at the mRNA or protein, or an increase at the activity level. Typically, the level of increase is at least 30%, 50%, 75%, 80%, 100%, 2-fold, 3-fold, 5-fold, 10-fold or more compared to the parent cells that have not be modified. The GenBank Accession Nos. for the E4BP4 and IFNG coding sequences are U83148 and V00543, respectively.

As used here, the term “natural killer cells” or “NK cells” is used to refer to a type of cytotoxic lymphocytes or effector cells of innate immunity. The human NK cells are characterized as expressing cell surface antigens CD16 and CD56 but without pan T marker CD3, T-cell antigen receptors (TCR), or surface immunoglobulins (Ig) B cell receptors. NK cells have the unique ability to detect and kill stressed cells (e.g., cells infected by virus or cancerous cells): rather than relying on antibodies or major histocompatibility complex (MHC), NK cells detect and kill compromised cells such as virus-infected cells or tumor cells upon activation by “missing self” or altered/diminished MHC on such cells.

### DETAILED DESCRIPTION OF THE INVENTION

### I. Introduction

Cancer remains one of the leading causes of human deaths. Cancer treatment with cytotoxic drugs is, however, frequently ineffective and presents high cytotoxicity with severe systemic side-effects. Increasing evidence shows that transforming growth factor-β (TGF-β) acts as a potent tumor promoter in established carcinoma. Cancer-derived TGF-β drives malignant progression by constitutively inducing epithelial to mesenchymal transition and tumor-associated angiogenesis, and by suppressing anti-tumor immunity in cancer microenvironment. Based that information, many therapeutic approaches by targeting TGF-0 receptors using soluble TGF-β receptor II, small molecule ALK5 kinase inhibitors, as well as neutralizing antibodies, have been developed by researchers and pharmaceutical companies. Some of them have been shown promise in early pre-clinical studies, including SD-093, SD-208, and SM16. However, TGF-β is a fundamental anti-inflammatory cytokine and general blockade of TGF-β at the level of TGF-β receptor is also problematic due to the likelihood of causing autoimmune diseases. Thus, research into the downstream of TGF-β signaling to identify more specific therapeutic targets related to cancer progression may offer a better anticancer therapy clinically.

The inventor's research group previously observed that mice null for Smad3, a key downstream mediator of TGF-β signaling, are protected against cancer growth, invasion, metastasis (for example, to lymph nodes, liver, lung, gastric, and colon tissues), and death in two highly invasive cancer models including lung carcinoma (LLC) and melanoma (B16F10). This finding indicates that Smad3-dependent cancer microenvironment in the host determines the cancer progression or regression. This also indicates that targeting Smad3 on the cancer microenvironment (as well as cancer) may offer a better anticancer therapy. The present invention provides an innovative method for a more effective cancer treatment strategy by using a genetic engineering TGF-β tolerant human NK-92 cells, namely a line of Smad3 knockdown human NK-92 cells (NK92-S3KD). These cells, while having significantly reduced Smad3 activity, exhibit augmented cancer-killing activity. This invention provides many advantages over the current anti-cancer treatments by largely enhancing the cancer-killing activities of NK cells and offers a safer and more effective means of immunotherapy for cancer.

### II. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a gene of interest, a polynucleotide encoding a polypeptide of interest, and synthetic oligonucleotides can be verified after cloning or subcloning using, e.g., the chain termination method for sequencing double-stranded templates of Wallace et al., Gene 16: 21-26 (1981).

### III. Smad3-Knockdown Cells

The present inventor revealed in previous studies that Smad3-TGFβ signaling plays a critical role in tumorigenesis. Specific targeting Smad3 for its suppression in the cancer microenvironment is thus recognized a potentially effective anti-cancer therapy. The present disclosure provides an innovative strategy in cancer treatment involving the use of an engineered TGF-β tolerant human NK-92 cell line, in which the endogenous Smad3 expression and thus activity is knocked down, i.e., either significantly inhibited or completely eliminated.

Suitable cells for human manipulation for targeted Smad3 suppression include various types of immune effector cells, including all T cell subsets, macrophages, dendritic cells, neutrophils, eosinophils, and mast cells, as well as non-immune cells including stem cells or fibroblasts. Suitable cells for such manipulation may be naturally occurring cells as well as artificially engineered or recombinantly produced cells, for example, genetically modified cells such as chimeric antigen receptor (CAR) T cells.

A Smad3-knockdown cell may be generated by genetic manipulation of the genomic Smad3 sequence of a suitable parent cell. Methods such as sequence homology-based gene disruption methods utilizing a viral vector or CRISPR system can be used for altering the Smad3 genomic sequence, for example, by insertion, deletion, or substitution, which may occur in the coding region of the gene or in the non-coding regions (e.g., promoter region or other regulatory region) and which may result in complete abolition of Smad3 expression, reduced Smad2 expression, or unaltered expression at mRNA level but diminished Smad3 protein activity.

Alternatively, Smad3-knockdown cells may be generated by introducing into suitable parent cells an exogenous expression cassette encoding (1) one or more polynucleotide sequence that can interfere with or inhibit the expression of Smad3 gene at mRNA level; or (2) a protein that can suppress the activity of Smad3 protein. For instance, an vector (such as a viral vector based on a viral genome structure) comprising at least one coding sequence for an siRNA, a microRNA, a miniRNA, a lncRNA, or an antisense oligonucleotide that is capable of disrupting Smad3 expression at the mRNA level may be used. As another possibility, the vector may introduce into the recipient cell one or more coding sequence encoding for a protein product that interferes with the biological activity of Smad3 and thus acts as an inhibitor of Smad3 protein. Some examples of such protein inhibitors include a neutralizing antibody against Smad3, a peptide that can bind and inactivate the Smad3 protein, or a dominant negative mutant of Smad3 protein. Any of the above-described exogenous sequences may be transiently present in a recipient cell or may be integrated into the recipient cell's genome thus present in a permanent manner.

Upon introducing of the exogenous polynucleotide sequence(s) into parent cells, the cells can be screened for evidence of suppressed Smad3 expression and/or activity. Various assays including polynucleotide detection assays (e.g., PCR or RT-PCR), immunological assays (e.g., western blot), and Smad3 functional assays (e.g., TGF-β stimulation assay) may be performed to identify desirable transformants exhibiting significantly diminished or abolished Smad3 expression and/or activity. Ideally, the level of decrease in Smad3 expression and/or activity is at least a 10% decrease compared to unmodified parent cells; more preferably, the decrease is at least 20%, 30%, 40%, 50%, 60%, 70%, 80%, or 90% or even greater including complete elimination.

In addition, since the present inventor illustrated for the first time the underlying inhibitory mechanism of SMAD3 in E4BP4-dependent anticancer activity of human mature NK cells including identifying a binding site of SMAD3 protein on the 3′ UTR of human E4BP4 (NFIL3) genomic sequence, as well as identifying a binding site of E4BP4 protein in the promoter region of human IFNG genomic sequence, an alternative to the Smad3-knockdown cells of this invention that may provide the same or similar anti-cancer utility is E4BP4- or IFNG-enhanced cells, especially NK-cells and other cell types named in this section. Various methods can be employed to achieve increased E4BP4 or IFNG expression and/or activity, such as by introducing extra copies of the E4BP4 or IFNG gene (e.g., by using a vector such as a viral vector carrying the extra copies) or by replacing the endogenous promoter(s) with exogenous, more potent promoter(s) for the E4BP4 or IFNG gene in the cells. Assays at the mRNA and/or protein level can be performed to confirm increased E4BP4 or IFNG expression and/or activity in the cells.

### IV. Cancer Therapy Using Modified Cells

The present invention also provides pharmaceutical compositions or physiological compositions comprising the Smad3-knockdown cells, preferably in an effective number such as in a number appropriate for dosing in a predetermined administration schedule. Such pharmaceutical or physiological compositions typically include one or more pharmaceutically or physiologically acceptable excipients or carriers. Pharmaceutical compositions of the invention can be formulated so as to be suitable for use in a variety of delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249: 1527-1533 (1990).

The pharmaceutical compositions of the present invention can be administered by various routes, e.g., by injection for systemic or local delivery. The preferred routes of administering the pharmaceutical compositions are subcutaneous, intramuscular, intravenous, intraperitoneal, and intratumoral injection. The appropriate dose may be administered in a single daily dose or as divided doses presented at appropriate intervals, for example, once per day, or once or twice per week, or once or twice per month. The dosing range of cells can vary from low dose (0.1-1.0×10⁶ cells/kg/dose) to moderate (0.1-1×10⁷ cells/kg/dose) and to higher dose (0.1-1×10⁸⁻⁹ cells/kg/dose). A single dose to multiple doses can be repeatedly used for at least one month to three, four, five, six, or nine months, or to one or more years and can be used alone or combined with other anti-cancer therapies such as chemotherapy, chemotherapy, or other immunotherapy.

For effectively treating a cancer patient, the pharmaceutical composition containing Smad3-knockdown cells may be administered concurrently with one or more additional therapeutic agents known to provide benefits for treating cancers or for alleviating the symptoms of cancers.

### Examples

The following examples are provided by way of illustration only and not by way of limitation. Those of skill in the art will readily recognize a variety of non-critical parameters that could be changed or modified to yield essentially the same or similar results.

Natural killer (NK) cell is the first-line anti-cancer immunity. However, it is paralyzed by a cancer-derived immunosuppressive cytokine TGF-β1. The present inventor recently discovered that the NK cell immunity against cancer is largely enhanced in mice lacking Smad3, a key downstream mediator of canonical TGF-β1 signaling. It is reported in this disclosure that genetically engineered a stable Smad3-knockdown human NK cell line NK-92-S3KD largely enhanced its anti-cancer effect on two xenograft mouse models of human hepatoma (HepG2) or melanoma (A375) by promoting the cancer-killing activity and production of INF-γ, granzyme B, and perforin. Mechanistically, the inventor identified that INFG is a novel target gene of transcriptional factor E4BP4 in the mature NK cells in which TGF-β1 suppresses NK cell differentiation and functions via the SMAD3-E4BP4 axis. Thus, silencing SMAD3 defected immunosuppressive effect of TGF-β1 on NK cells and therefore restored the E4BP4-dependent INF-γ production and NK cell anti-cancer activities. In conclusion, a TGF-β1 tolerant human natural killer cell line has been successfully developed for effective anticancer immunotherapy by silencing Smad3. The SMAD3-silencing human NK cell line may represent as a novel and effective immunotherapy for cancer clinically.

### Introduction

Cancer is still one of the leading causes of death in the world. Traditional strategies including surgery, chemotherapy and radiotherapy have been used as the mainstay treatments of cancer for decades clinically; however, outcomes are often still unsatisfactory due to severe side effects, drug resistance, recurrence, and metastasis. Indeed, cancer cells are high in heterogeneity and versatility, therefore eventually adapt to the external environments and lead to primary and secondary resistance (1). In addition, the severe side effects of systematic anticancer treatments using cytotoxic drugs are also a serious problem clinically (2). Recently, targeting tumor microenvironment is a new therapeutic approach for cancer, as tumor growth, invasion, and metastasis largely rely on their stromal conditions (3). Indeed, the cell-based immunotherapies including cytotoxic T lymphocytes (CTL) and natural killer (NK) cells have showed considerable progresses in clinical practice (4-6).

While results from clinical studies of NK cell adoptive therapy are encouraging (7-11), NK cell-based cancer immunotherapy is recently suggested as a promising therapeutic option for solid tumors. Several studies demonstrated that the quantity of intratumoral NK cells is negatively correlated with the tumor progression (12,13). However, application of NK cell-based therapies in solid tumor is still in challenge due to the secretion of immunosuppressive cytokines and downregulation of activating ligands in the microenvironment of solid tumors (14,15).

Accumulating evidence shows that TGF-β1 is largely produced by cancer cells and promotes cancer progression by greatly restricting or paralyzing the function of immune cells against cancer (16). It is now clear that TGF-β1 acts as a potent promoter at the progressive phase of tumorigenesis to trigger the malignant progression by inducing epithelial to mesenchymal transition (EMT), tumor-associated angiogenesis, as well as suppressing anti-cancer immunity in the tumor microenvironment. In addition, TGF-β singling can suppress cytolytic activity of NK cells via down-regulating interferon responsiveness and CD16-mediated interferon-gamma (IFN-γ) production in vitro (17-19). Thus, targeting TGF-β signaling in the tumor microenvironment with TGF-β neutralizing antibody, antisense oligonucleotide, and TGF-β receptors inhibitors become new strategies for eliminating cancers (20-26). However, completely blockade of TGF-β signaling will cause autoimmune diseases due to its anti-inflammatory features as evidenced by the development of adverse side effects including systemic inflammation, cardiovascular defects and autoimmunity in mouse models (27). Thus, identification of a precise and accessible therapeutic target in the downstream of TGF-β signaling should offer a better clinical outcome for the anti-cancer treatment.

It was recently revealed that Smad3, a downstream mediator of TGF-β singling (28), is essential for tumor microenvironment to promote tumor growth, invasion, and metastasis in mice. Genetic deletion or pharmacological inhibition of Smad3 dramatically prevents the lethal progression of both lung carcinoma and melanoma by enhancing the NK cell cancer-killing activities and the production of NK cells in the tumor microenvironment (29). These findings suggested that Smad3 is a novel therapeutic target for eliminating the TGF-β-mediated immunosuppression in tumor microenvironment. Thus, the present work aims to translate our research findings into clinical application via developing a NK cell-specific SMAD3-targeted therapy. As disclosed here, the inventor genetically engineered a stable SMAD3-knockdown human NK cell line (NK-92-S3KD). Treatment with NK-92-S3KD produced better anticancer effects than its parental cell line on NOD/SCID mice bearing human hepatoma (HepG2) or melanoma (A375) in vivo. Mechanistic study uncovered that knockdown of SMAD3 enhanced cancer-killing activities of mature human NK cells via blocking the TGF-β1/SMAD3/E4BP4 inhibitory axis. As the parental cell line NK-92 is already enrolled in clinical trials, this novel NK92-S3KD will further advance the anticancer efficiency of NK cell based immunotherapy clinically.

### Results

### Knockdown of SMAD3 Largely Enhances the Cancer-Killing Activity of Human NK Cell Line NK-92 In Vitro.

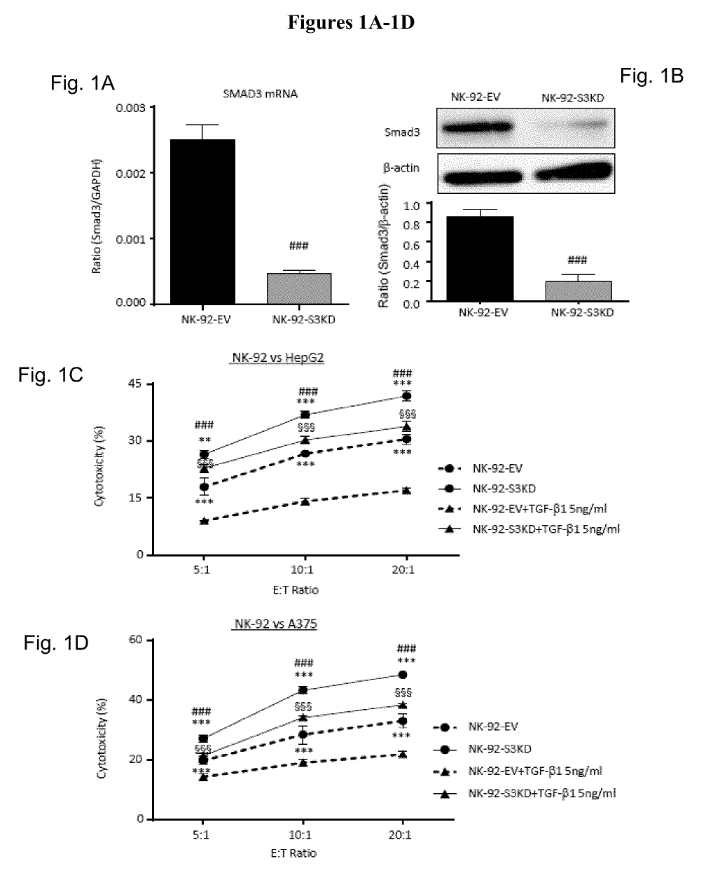

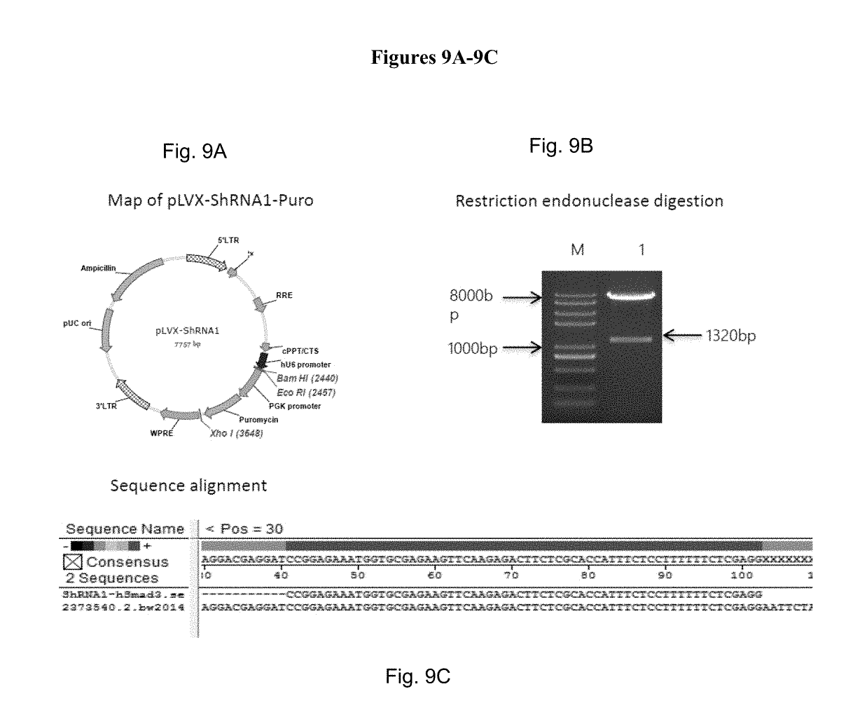

To examine the functional role of SMAD3 in NK cell anti-cancer activity, the inventor first developed a stable SMAD3-knockdown human NK cell line by transducing NK-92 cells with a lentivirus containing shRNA specifically against human SMAD3 mRNA (shRNA-hSmad3) (FIG. 9). Real-time PCR demonstrated that shRNA-hSmad3 transduction largely down-regulated mRNA expression of SMAD3 in NK-92 cells (FIG. 1A), which was further confirmed by western blot analysis in which more than 70% decrease in SMAD3 protein was detected (FIG. 1B). Reduction of SMAD3 in the clonally selected shRNA-hSmad3 transduced NK-92 cells was maintained for more than six months and a stable SMAD3-knockdown NK-92 cell line (NK-92-S3KD) was successfully developed.

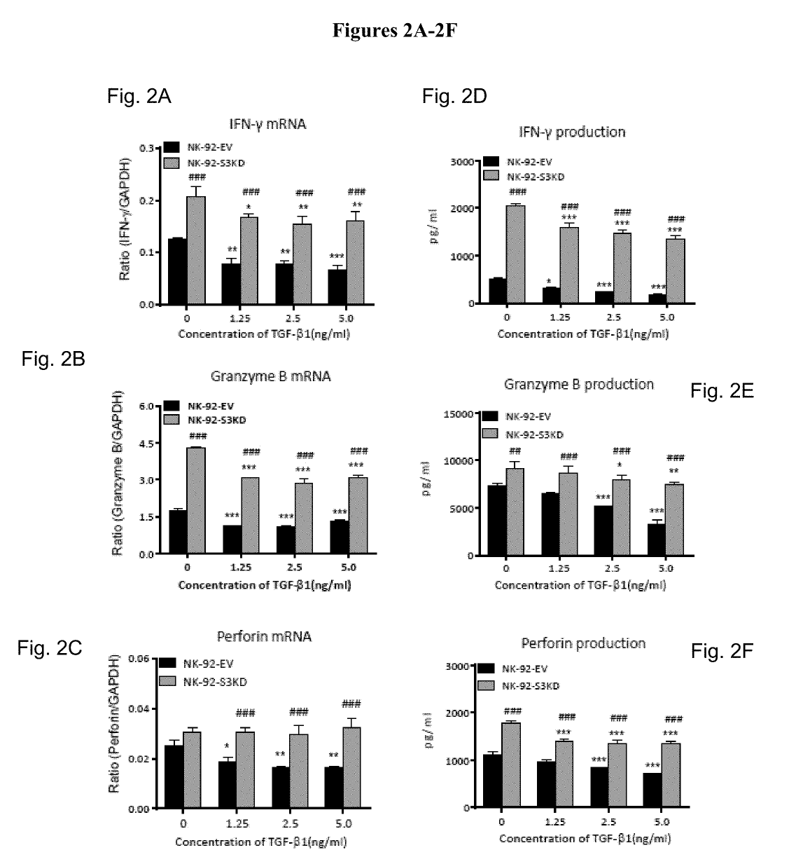

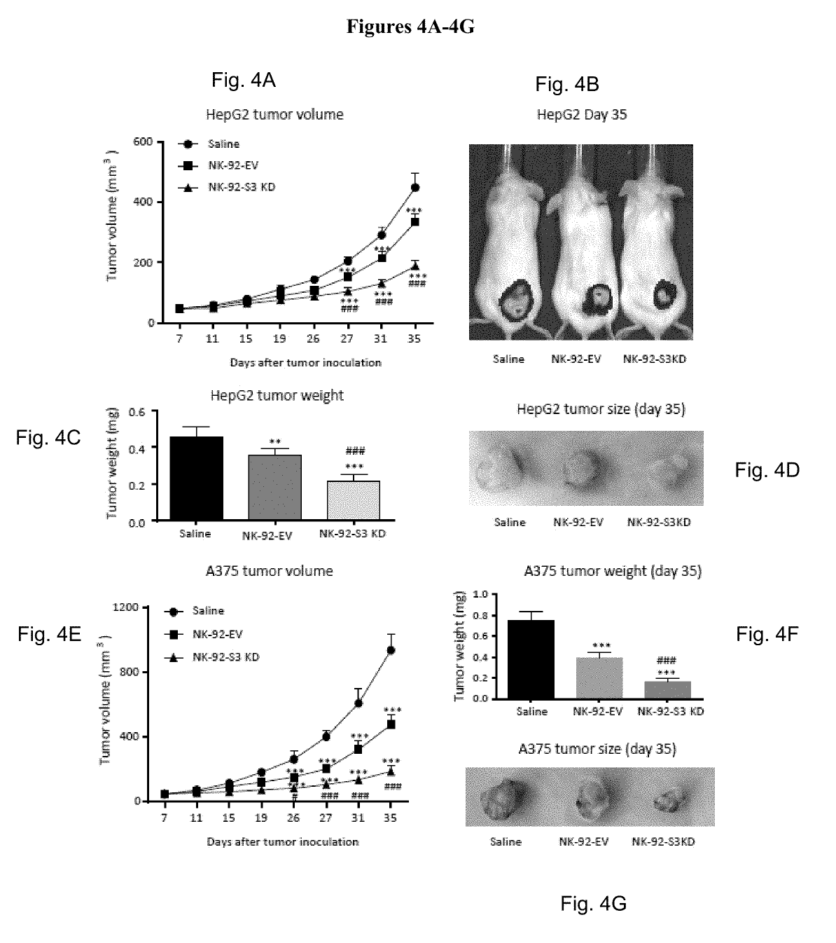

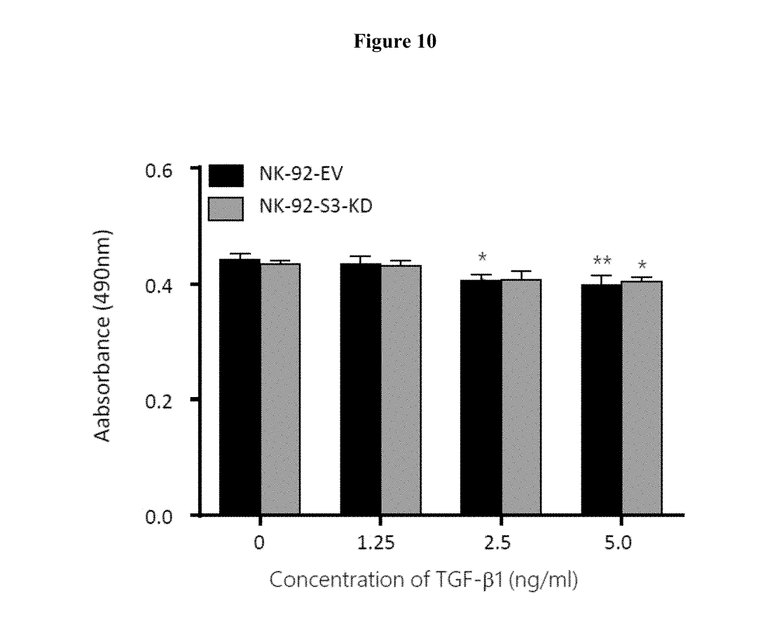

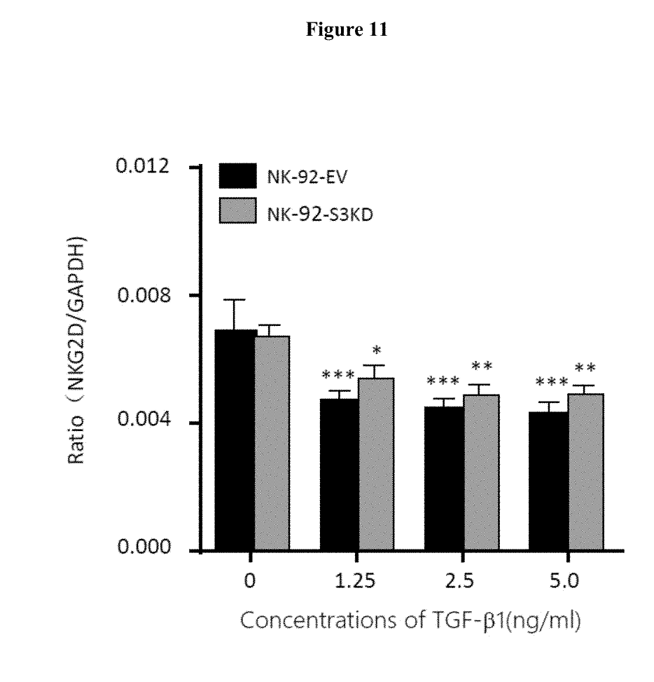

The anticancer effects of NK-92-S3KD against human hepatoma and melanoma cells was then tested by LDH release assay in vitro. As shown in FIGS. 1C and D, knockdown of SMAD3 largely improved the cancer-killing activities of NK-92 cells. To mimic the tumor microenvironment with high TGF-β1 conditions, TGF-β1 at a dose of 5 ng/ml was added into the culture. As expected, addition of TGF-β1 significantly inhibited the cancer-killing capacity of NK-92-EV cells (empty vector control) against HepG2 and A375 cells in various E/T ratios. Strikingly, stable knockdown of SMAD3 markedly enhanced the cytotoxicity of NK-92-S3KD cells under high TGF-β1 conditions (FIGS. 1C and D). In addition, real-time PCR and ELISA also revealed that TGF-β1-mediated suppression of anticancer cytokines (i.e., IFN-γ, Granzyme B, and Perforin) was attenuated in NK-92-S3KD when compared with the NK-92-EV cells (FIG. 2). Similarly, FIG. 4 shows the effect of Smad3 suppression in the NK cells on GM-CSF expression in response to TGF-β: exposure to TGF-β1 suppressed GM-CSF expression at both mRNA and protein levels, which was prevented by suppression of Smad3 in HK92-S3KD cells. These results clearly demonstrated that stable knockdown of SMAD3 in mature human NK cells was capable of attenuating the TGF-β1-mediated immunosuppression and therefore largely enhanced the cancer-killing effect and anticancer cytokine production in the NK-92-S3KD cells. However, knockdown of Smad3 did not influence NK-92 proliferation and differentiation as determined by the MTT assay and expression of NKG2D (FIGS. 10 and 11).

Treatment with NK-92-S3KD Suppresses Cancer Progression in Human Hepatoma (HepG2) and Melanoma (A3 75)-Bearing Mice

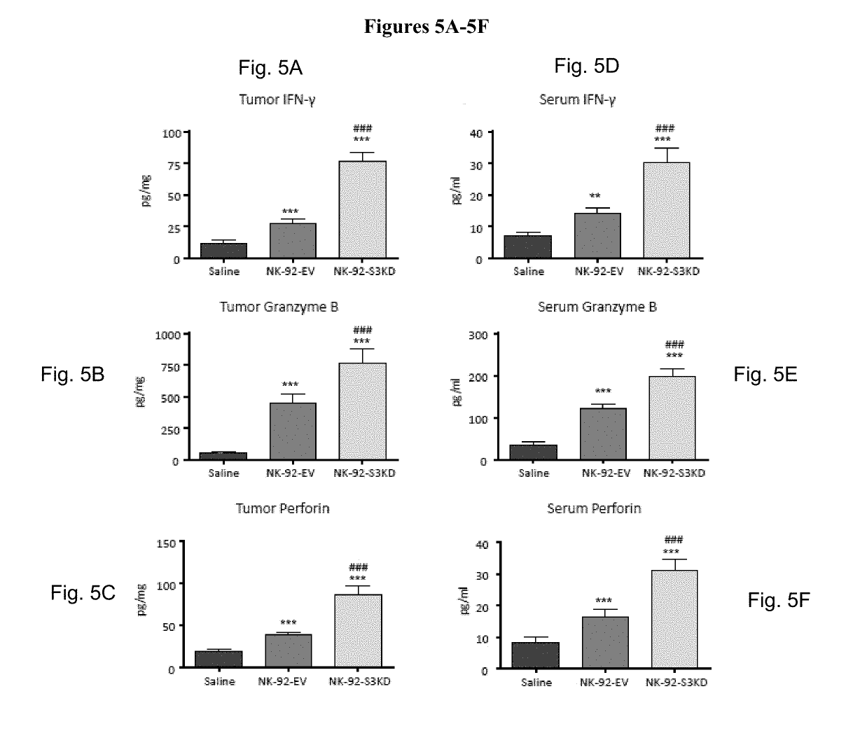

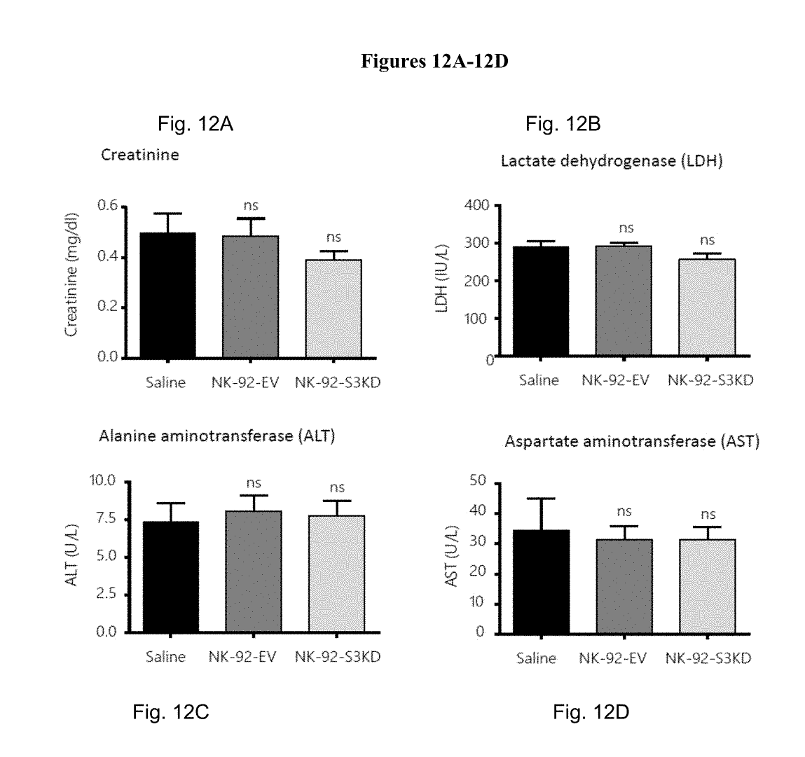

To assess the anti-tumor effect of NK-92-S3KD in vivo, xenografts tumor models of human hepatoma (HepG2) and melanoma (A375) were generated on NOD/SCID mice in which the host NK cells are deficient. On day 7 after subcutaneous tumor inoculation, the HepG2- or A375-tumor bearing mice were treated with saline, NK-92-EV or NK-92-S3KD cells (2×10⁷ cells/mouse) twice a week with IL-2 administration (200 ng/mouse) every other day. Treatment with NK-92-EV cells effectively inhibited the growth of HepG2 and A375 tumors as determined by the tumor volume, which was further suppressed in those received NK-92-S3KD cells (FIGS. 4A and 4E). Similarly, treatment with NK-92-EV significantly reduced the size and weight of HepG2 and A375 tumors on day 35, which was further reduced in the NK-92-S3KD treatment group (FIGS. 4B-D and F-G). In line with the in vitro findings, as shown in FIG. 5, treatment with NK-92-S3KD cells greatly increased both intratumoral and serum levels of IFN-γ, granzyme B, and perforin in the HepG2-tumor bearing mice compared with the saline—as well as the empty vector-controls; clearly demonstrating that disruption of SMAD3 largely enhances the anti-cancer activities of mature NK cell in vivo. Furthermore, treatment with NK-92-EV or NK-92-S3KD did not cause adverse side effect on kidney, heart and liver since no significant changes in the serum levels of creatinine, lactate dehydrogenase (LDH), alanine aminotransferase (ALT) and aspartate aminotransferase (AST) was detected in both of the treated mice on day 28 compared with the saline controls (FIG. 12). Thus, NK-92-S3KD may represent as a novel, safe and effective anticancer immunotherapy for cancer.

### Targeting SMAD3 Enhances E4BP4-Dependent Cancer-Killing Activities in the Mature NK Cells

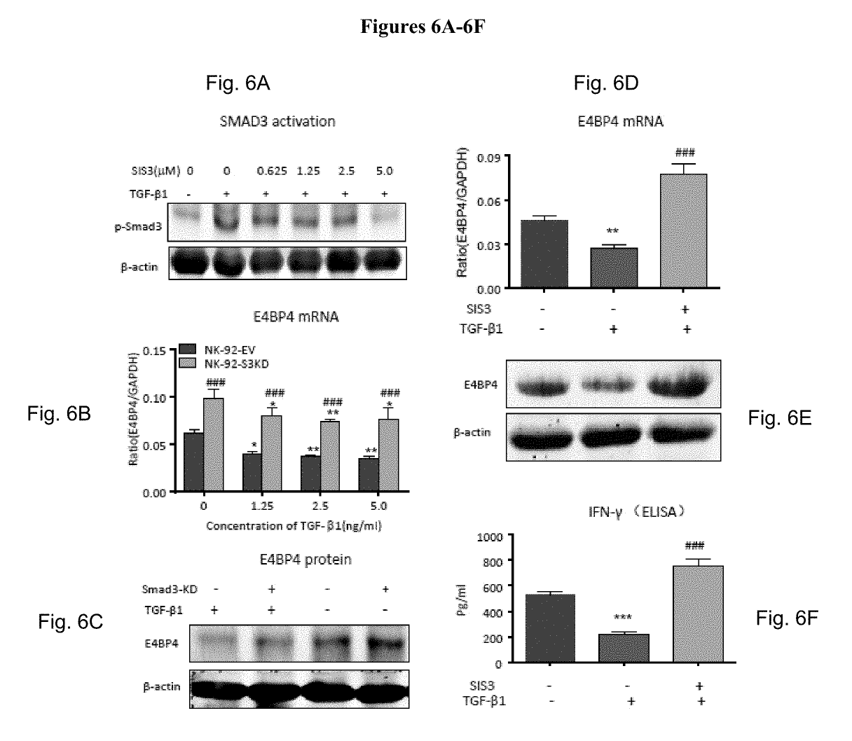

The potential mechanism whereby disruption of Smad3 promoted anticancer activities of the mature human NK cells was next examined. It was recently revealed that TGF-β1 can suppress the murine NK cell differentiation via a Smad3/E4BP4-dependent mechanism (29), but its potential role in the mature NK cells is unknown. In this study, the regulatory role of the TGF-β1/Smad3/E4BP4 axis in the activity of mature NK cells was further examined. As shown in FIGS. 6A and 6B, real-time PCR and western blot detected that addition of TGF-β1 (5 ng/ml) was able to induce phosphorylation of Smad3 and inhibition of E4BP4 mRNA expression in a dosage-dependent manner. Blockade of Smad3 with Smad3 inhibitor (SIS3) (30) or viral-mediated knockdown (Smad3-KD) resulted in a markedly increase in the expressions of E4BP4 mRNA and protein as well as production of IFN-γ in the NK-92 cells under high TGF-β1 condition (FIG. 6 D-F). These findings demonstrated an essential role of TGF-β1/Smad3/E4BP4 axis in the activity of mature NK cells.

### IFNG is an E4BP4 Target Gene Regulated by SMAD3 in Mature Human NK Cells

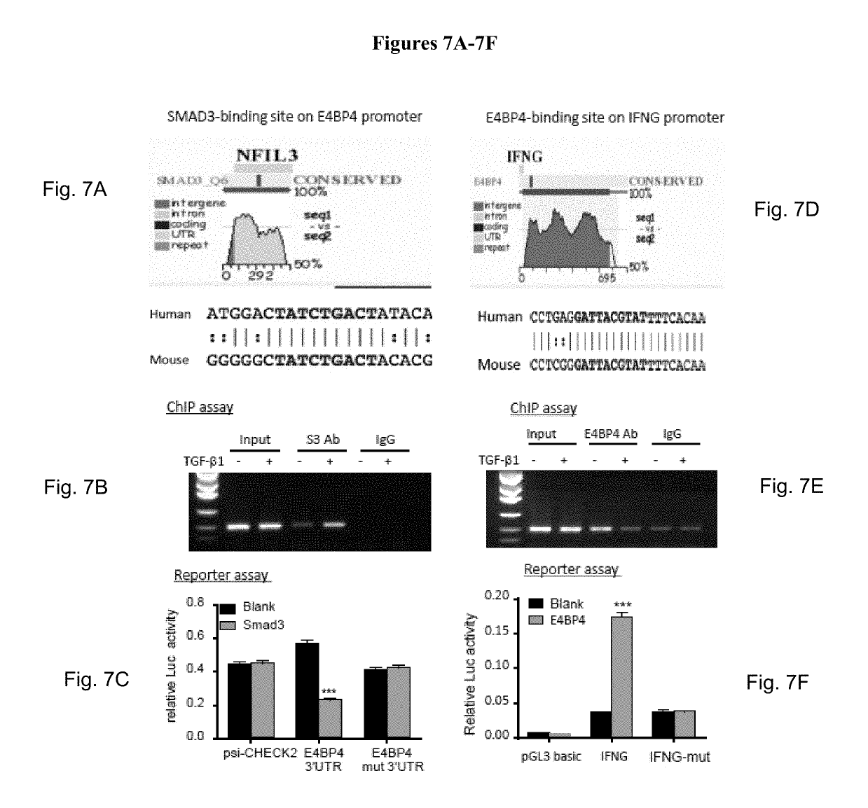

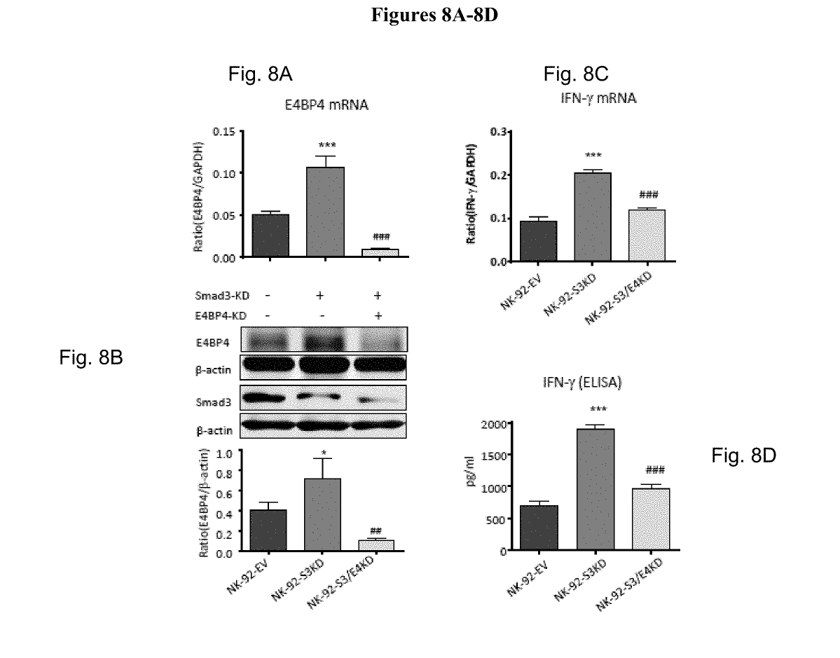

The transcription factor E4BP4 was first discovered in NK cell differentiation (31), however, its role and regulatory mechanism in mature NK cells is still largely unexplored. In this study, the underlying inhibitory mechanism of SMAD3 in E4BP4-dependent anticancer activity of human mature NK cells was further elucidated by identifying a binding site of SMAD3 protein on the 3′ UTR of human E4BP4 (NFIL3) genomic sequence (FIG. 7A). Indeed, ChIP and luciferase reporter assays revealed that TGF-β1 promoted the physical binding of SMAD3 protein on 3′UTR of E4BP4 gene, therefore inhibiting the transcription of E4BP4 (FIGS. 7B and C). More importantly, a binding site of E4BP4 protein is further predicted on the promoter region of human IFNG genomic sequence by ECR browser (32) (FIG. 7D). Interestingly, TGF-β1 largely suppressed the binding of E4BP4 proteins on the IFNG promoter as shown in FIG. 7E. Thus, TGF-β1 was capable of inhibiting the promoter activity of IFNG by reducing the availability of E4BP4 proteins via the TGF-β1/Smad3/E4BP4 inhibitory axis, thereby blocking the transcription of IFNG gene in the NK-92 cells (FIGS. 7E and F). The dual-luciferase reporter assays showed that mutation of the SMAD3 or E4BP4 binding sites abrogated their transcriptional regulatory effects on the E4BP4 or IFNG promoter activities respectively (FIGS. 7C and F). This was further confirmed in vitro by silencing SMAD3 to significantly increase the mRNA and protein expression levels of E4BP4 and IFN-γ in the NK-92 cells (NK-92-S3KD) in an E4BP4-dependent manner as determined in double Smad3 and E4BP4 knockdown NK cells (NK-92-S3/E4KD) (FIG. 8). Thus, these findings clearly demonstrated the direct regulatory mechanism of Smad3/E4BP4/IFNG axis in the mature NK cells. Therefore targeting Smad3 restores IFN-γ production of the human mature NK cells under TGF-β1-mediated immunosuppression in the tumor microenvironments.

### Discussion

Targeting tumor microenvironment is a new strategy for eliminating cancer. Increasing evidence shows that NK cell-based innate immunotherapy is more accessible due to its unique features (e.g., antigen-independent, non-MHC restricted, no prior immunization require, and less possibility of inducing GVHD) (33,34), compared with the limitations of T cell-based adaptive immunotherapy (35). However, the outcomes of clinical trials using the NK cell-based adoptive cellular therapy are inconsistent (36,37). In this study, the present inventor significantly improved the cancer-killing effects of a clinical trial enrolled human NK cell line NK-92 by targeting SMAD3 (NK-92-S3KD). These findings showed that SMAD3-knockdown largely enhances the anti-cancer effects of NK cells without significant side effects in vivo. Mechanistically, depletion of SMAD3 circumvents the inhibitory effects of TGF-β1 on the E4BP4-IFNG axis in mature NK cells, thereby promoting anticancer cytokine production in the tumor microenvironment. Thus, this work developed a novel strategy to overcome TGF-β1-mediated immunosuppression that may represent as an effective SMAD3-targeted immunotherapy for cancer clinically.

Recently, researchers made a lot of attempts to enhance the anti-cancer effects of NK cell-based immunotherapy. Nagashima and Imamura demonstrated that stable IL-2 and IL-15 expressions increases anti-cancer responses of NK cell immunotherapy respectively (38,39). Other strategies for enhancing NK cell mediated cytotoxicity include overexpressing NK activating receptor NKG2D, down-regulating NK inhibitory receptor NKG2A and delivering high affinity CD16 (HA-CD16) gene to NK cell (40-42). Somanshi et al. focused on strengthening the migration ability via genetically delivering CCR7 in NK cell (43). Besides, some researchers focused on improving NK cell capability of tumor-recognition and activation via transducing chimeric antigen receptors (CARs) targeting various tumor antigen such as CD19, CD20, Her2/Neu, ErbB2, CEA, GPA7, EpCAM (44-50). Unfortunately, all these works cannot prevent the fact that cancer cell-derived TGF-β1 can largely suppress the anticancer effects of NK cells in multiple aspects including proliferation, maturation, cytokine production, as well as receptor activation (51-53). Therefore, the modified NK cells are still paralyzed in the TGF-β1-rich tumor microenvironment. Accumulating evidence demonstrated that TGF-β1 inhibits IFN-γ production in NK cells, although the underlying mechanism is still largely unexplored. It is reported that TGF-β1 regulates IFN-γ expression via a Smad3-dependent signaling by directly binding on the promoter region of IFNG as a transcriptional suppressor or indirectly suppressing T-BET (54, 55). More importantly, the present work revealed a novel mechanism for TGF-β/Smad3-mediated IFN-γ suppression by transcriptionally suppressing E4BP4, a master transcription factor for NK cell development (56). In the present work, the inventor also identified a novel SMAD3/E4BP4/IFNG inhibitory axis for TGF-β1-mediated NK cell suppression. Hence, targeting this inhibitory axis by inactivating the TGF-β/SMAD3 signaling pathway on NK cells may represent a novel and effective immunotherapy for cancer clinically.

So far, only one study suggested the development of TGF-β tolerant NK cell line (57), in which TGF-β signaling pathway was blocked specifically in NK-92 cell via genetically overexpressing a dominant negative TGF-β receptor II. The enhanced anti-cancer activity of this TGF-β insensitive cell line was demonstrated on Calu-1 cell bearing nude mice. However, the role of non-canonical pathway in NK cell activity is largely unknown; indiscriminately blocking TGF-β at the receptor level may also cause unfavorable immune response on NK cells. It is known that T cells isolated from Smad3-deficient mice are resistant to TGF-β1 inhibition (58). On the contrary, overexpression of Smad3 increases the sensitivity to inhibitory effects of TGF-β in NK cell (19,54). Thus, the development of this novel TGF-β1 tolerant NK cell line by targeting Smad3 may provide more specific and effective immunotherapy for cancer with many advantages over targeting on the TGF-β receptor levels.

Here, the clinical trial enrolled NK cell line NK-92 for gene manipulation was selected based on several reasons. First, in comparison with primary NK cell, NK-92 cell line is more practical for large-scale expansion and quality control. Second, NK-92 cell induces less KIR-MHC I dependent inhibition due to the lack of inhibitory KIRs. Third, the lack of immunogenicity in this cell line results in less opportunity of being rejected by the immune system of recipients (59). Besides, as an adoptive effector cell widely tested in clinical trials, the safety of NK-92 cell can be guaranteed to a certain extent (60,61). Genetic modification has been widely used as promising strategy for improving anti-cancer effects of T cells (62,63). However, limited genetic manipulation has been carried out in NK cells due to the technical challenges of gene transfer (64). The unstable efficiency of gene delivery in NK cell lines with lentiviral transduction ranges from 2-97%, multiple rounds of virus transduction may be required in some cases (50,65). In order to stably down-regulating SMAD3 expression in NK-92 cells, recombinant lentivirus was used in the present study. The shRNA targeting SMAD3 mRNA was successfully delivered into NK-92 cells with recombinant lentivirus, and eventually integrated the sequence encoding SMAD3 shRNA into host genome. The expression of SMAD3 protein was stably knockdown in NK-92 cells (NK-92-S3KD), which exhibits the property of tolerance to TGF-β1 and enhanced anti-cancer effects in vitro and in vivo. For the potential feasibility of clinical application, safety of using lentiviral vector in clinical setting must be considered. Up to present, at least 40 clinical trials using lentiviral vectors have been approved. Gerard J. McGarrity et al. followed 263 infusions of lentivirus-transduced cells to assess the safety of lentivirus vector, part of subjects have been followed for over 8 years, no obvious adverse events were observed during the follow-up (66). In this study, no obvious organ damage was detected in the tumor bearing mice receiving NK-92-S3KD cells. The newly developed NK-92-S3KD cell line may facilitate the future clinical application in terms of adoptive immunotherapy.

In order to minimize the influence of immune system from the tumor bearing mice, NK and T cells deficient NOD/SCID mice were employed in this study. However, the anti-cancer effects of NK-92-S3KD cells may be underestimated in this xenograft model. Indeed, these data demonstrated that disruption of SMAD3 significantly enhances the production of IFN-γ whose anti-cancer effects are at least partially depend on the activation of macrophage (67) and cytotoxic T cell (68). Unfortunately, absence of T cell and deficiency of macrophage are unique features of the NOD/SCID mice (69,70). Therefore, further verifying the anti-cancer effects of NK-92-S3KD cell using a humanized mouse tumor model may be necessary before application in clinical trial.

In conclusion, this is the first work to generate a TGF-β tolerant NK-92 cell line via genetically targeting Smad3. An enhanced anti-cancer activity in this NK-92-S3KD cell line is clearly demonstrated in mice. A novel TGF-β1 mediated SMAD3/E4BP4/IFNG inhibitory axis is identified in mature human NK cells as well. This novel TGF-β tolerant NK-92 cell line may represent a promising immunotherapy for cancer clinically.

### Materials and Methods

### Antibodies, Cell Lines and Mice

Antibodies used in this study were listed in Table 1. Human NK-92 cell line, human A375 cell line and 293T cell line were obtained from American Type Culture Collection (ATCC). Human HepG2-Luc cell line was preserved in our laboratory. NOD/SCID (NOD.CB17-Prkdcscid/J) (6-8 weeks old) mice were purchased from the Jakson Laboratory (Stock No: 001303) and housed in a pathogen-free facility in microisolator cages, and fed with autoclaved food and water.

### Cell Culture

NK-92 cells were cultured in MEM alpha medium (Life Technologies), supplemented with 12.5% fetal bovine serum (Life Technologies), 12.5% horse serum (Rockland), 50 μmol/l β-Mercaptoethanol, 0.2 mmol/L inositol, 0.02 mmol/L folic acid, and 20 ng/ml of human rIL-2 (Life Technologies) in 5% CO₂ at 37° C. HepG2-Luc and A375 cells were cultured in DMEM/F12 medium (Life Technologies), supplemented with 10% fetal bovine serum, 1% penicillin and streptomycin in 5% CO₂ at 37° C. 293T cells were maintained in DMEM-High Glucose medium (Life Technologies) supplemented with 10% fetal bovine serum in 5% CO₂ at 37° C.

Construction of Recombinant Plasmid pLVX-shRNA1-Puro-hSMAD3

The vector PLVX-ShRNA1-Puro (Biowit Technologies) (FIG. 9A) was used as plasmid backbone in this experiment. This lentiviral vector allows the expression of interest gene and puromycin resistance gene. The cDNA sequence coding shRNA specially targeting human SMAD3 mRNA was listed in Table 3. The fragment was then cloned into the BamH I/EcoR Irestriction site of the backbone for construction of recombinant plasmid pLVX-shRNA1-Puro-hSMAD3. The accuracy of the inserted DNA fragment was identified by restriction enzyme digestion with Xho I and DNA sequencing.

Generation of Recombinant Lentiviral Particles rLV-hSMAD3

The recombinant plasmid pLVX-shRNA1-Puro-hSMAD3 was delivered into the packing cell 293T according to the manufacturer's instruction of lentivirus packaging kit (Biowit Technologies) to generate the recombinant lentiviral particles (rLV-hSmad3). Viral supernatants were harvested 48 hours after transfection and the titer of lentiviral particles were determined. The produced lentiviral particles were then stored at −80° C. for further use.

Gene Manipulation of SMAD3 in NK-92 Cells with rLV-hSMAD3

NK-92 cells were transduced with rLV-hSMAD3 and selected with puromycin (InvivoGen). Briefly, NK-92 cells were seeded in a 24-well plate at a density of 1×10⁶/ml and mixed with ploybrene (Santa Cruz) at final concentration of 5 ug/ml and rLV-hSmad3 at MOI (multiplicity of infection) equal to 50 overnight at 37° C. The transduced cells were then expanded in complete medium and selected with puromycin at a final concentration of 2 ug/ml. The expression level of SMAD3 in the puromycin resistant clone was determined by real-time PCR with corresponding primers (Table 2) and Western blot analysis with rabbit anti human SMAD3 antibody (Abcam) respectively.

### Cytotoxicity Assay

NK-92 cell mediated cytotoxicity was determined with 4 hours-lactate dehydrogenase (LDH) release cytotoxicity assays (CytoTox 96® Non-Radioactive Cytotoxicity Assay, Promega). Cytotoxicity against human hepatocellular carcinoma cells (HepG2) and malignant melanoma cell (A375) was measured at different effector/target (E/T) ratios of 5:1, 10:1 and 20:1 respectively. In brief, target cells were seeded in a 96-well plate at 1×10⁴ cells/well. The effector cells including NK-92-S3KD and NK-92-EV cells pretreated with or without 5 ng/ml TGF-β (R&D Systems) for 24 hours were co-cultured with target cells in indicated E/T ratios for 4 hours at 37° C. in 5% CO₂. LDH release in the co-culture supernatant, which is proportional to the number of lysed tumor cells, was determined by the absorbance at 490 nm wavelength. Cytotoxicity was evaluated with the following formula: % cytotoxicity=(Experimental−Effector Spontaneous−Target Spontaneous)/(Target Maximum−Target Spontaneous)×100.

### Real-Time RT-PCR

Total RNA from cells was isolated using the PureLink™ RNA Mini kit (Life Technologies) according to the manufacturer's instruction. The reverse transcription reaction was conducted with C1000 thermal cycler. The cDNA was then diluted with 40 ul RNase-free water and used as the template in real-time polymerase chain reaction. The relevant primer sets used are listed in Table 2.

### Enzyme-Linked Immunosorbent Assay (ELISA)

To measure the levels of cytokines in cell culture supernatants, tumor tissues and mouse serum, ELISA commercial kits for detection of human IFN-γ (BioLegend), Granzyme B (MABTECH) and perforin (Abcam) were used. Briefly, NK-92-EV and NK-92-S3KD cells (1×10⁶/ml) were cultured in 6-well plate in the presence or absence of TGF-β1 for 12 hours and the supernatants were collected for ELISA. For preparing the samples from tumor tissue, chilled PBS was added in tumor tissue samples at the ratio of 100 mg tissue per milliliter. Then the mixture was homogenized. By centrifuging at 14,000 rpm for 10 minutes at 4° C., the tumor tissue fluids were collected for ELISA. For preparing the samples of serum, tumor bearing mice were scarified on indicated day and mouse serum was collected via centrifuging the blood at 3000 rpm for 15 min at 4° C.

### MTT Assay

NK-92-EV cells or NK-92-S3KD cells were placed in the density of 1×10⁴/well in 96-well plate and treated with TGF-β1 for 44 hours. Subsequently, MTT (3-(4,5-dimethylthiazol-2-yl)-2,5-diphenyltetrazolium bromide, Invitrogen) was added in a final concentration of 0.5 mg/ml and incubated for 4 hours at 37° C. After disposal of the medium in the wells, DMSO was added to solubilize the formazan. The quantity of formazan, which represents the viability of cells, was recorded by the absorbance at a wavelength of 490 nm using a plate reading spectrophotometer.

### Adoptive Transfer of NK-92 Cells in Xenografts Tumor Models

Animal experiments were approved by the Animal Experimentation Ethics Committee of The Chinese University of Hong Kong (protocol no. 13/049/GRF). All handling and experimental procedures were carried out following experimental animal guidelines. Mice were subcutaneously inoculated with 5×10⁶HepG2-Luc or A375 cells. 7 days after tumor inoculation, when the tumor volume reached 50 mm³, the mice were assigned into 3 groups randomly. Saline, 2×10⁷NK-92-EV cells or equivalent number of NK-92-S3KD cells were injected into the mice intravenously at day 7, 10, 14, 17, 21, 24, 28 and 31 after tumor cell inoculation. All the mice were received rhIL-2 (200 ng/mouse) every other day via intraperitoneal injection. Tumor size were measured every four days and volume were calculated with following formula volume (v)=width (w)×length (1)×height (h)×πin. In vivo imaging system (IVIS) analysis was performed on HepG2-Luc bearing mice at day 35 after tumor inoculation. Mice were sacrificed at day 35 and tumors were weighed. Tumor tissue and mouse serum were collected for further studies.

### Measurement of Creatinine, Lactate Dehydrogenase (LDH), Alanine Aminotransferase (ALT) and Aspartate Aminotransferase (AST) Levels in Mouse Serum

Commercial kits including Stanbio-Creatinine LiquiColor® Test (Endpoint), ALT/SGPT Liqui-UV® Test (Rate) and AST/SGOT Liqui-UV® Test (Rate) which were purchased from Stanbio Laboratory were used in measurement of Creatinine, ALT and AST respectively. QuantiChrom™ Lactate Dehydrogenase Kit (DLDH-100) used for LDH detection was purchased from BioAssay. All the procedures were performed following instructions provided by manufacturers.

### Immunofluorescence

HepG2 bearing mice were sacrificed on day 35 after tumor inoculation and tumor tissues were collected for immunofluorescence staining. NK-92 cells infiltrated in tumor sites were identified by FITC-conjugated anti-human CD56 antibody. Cell nucleuses were counterstained with DAPI. The results were expressed as average proportion of CD56 positive cells in total DAPI positive cells.

Inhibition of SMAD3 with SIS3

To reveal the regulatory role of SMAD3 and E4BP4 in the production of IFN-γ in NK-92 cells, a specific inhibitor of SMAD3 known as SIS3 (Sigma) was used in this study. Briefly, NK-92 cells were pretreated with SIS3 at various concentrations for 2 hours. The cells were then treated with TGF-β1 at the final concentration of 5 ng/ml for 45 min and harvested for detection of the phosphorylation level of SMAD3 with western blot. The dosage of SIS3 that induced the maximum effect of phosphorylation inhibition of SMAD3 was determined as the optimal dosage of SIS3 used in further experiment. NK-92 cells were then pretreated with or without SIS3 at the determined concentration for 2 hours followed by stimulation with TGF-β1 at the final concentration of 5 ng/ml for 12 hours. The cells were harvested to detect the level of E4BP4 and IFN-γ.

### Knocking Down E4BP4 in NK-92-S3KD Cells

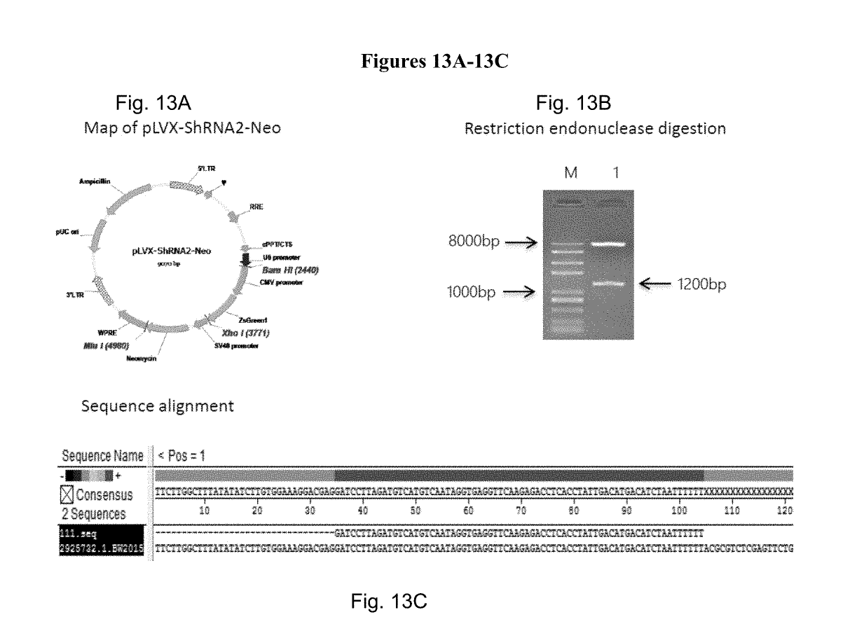

Similarly to genetic knocking down Smad3 in NK-92 cells, E4BP4 was knocked down in NK-92-S3KD cells. Briefly, NK-92-S3KD cells were transduced with recombinant lentivirus expressing shRNA targeting human E4BP4. The backbone used in the construction of recombinant plasmid is pLVX-ShRNA2-Neo (FIG. 13A). The cDNA sequence coding shRNA-E4BP4 was listed in Table 3. G418 (GENETICIN) was used for positive clone selection. The selected colony was then expanded and analyzed for E4BP4 expression level with real-time RT-PCR and western blot.

### Chromatin Immunoprecipitation (ChIP) Assay

To detect the physical binding of protein to special region of DNA, Chromatin Immunoprecipitation Assay (ChIP Assay) was performed with SimpleChIP® Enzymatic Chromatin IP Kit (Cell Signaling). 2×10⁷NK-92 cells were treated with or without TGF-β1 for 1 hour for SMAD3/E4BP4 ChIP Assay or 12 hours for E4BP4/IFNG ChIP Assay. ChIP Assay was performed following manufacturer's instructions. The Rabbit anti human antibodies used in ChIP Assay were listed in Table 1. The primer sets were designed based on the predicted binding site provided by ECR Browser database and listed in Table 2.

### Dual Luciferase Reporter Assay

To evaluate whether the physical protein-DNA binding can induce measurable regulatory effects, Dual Luciferase Reporter Assays were performed. Briefly, for Smad3/E4BP4 reporter assay, CDS (coding sequence) region of human SMAD3 was amplified and cloned into pcDNA3.1+ vector to construct SMAD3 expressing plasmid pcDNA3.1+SMAD3. Then, a reporter plasmid was constructed expressing E4BP4 3′UTR with psi-CHECK2. Furthermore, the predicted binding site TATCTGACT was mutated and plasmid expressing mutant of E4BP4 3′UTR was obtained. Similarly, for E4BP4/IFNG reporter assay, CDS region of human E4BP4 was cloned into pcDNA3.1+. The IFNG promoter was cloned into vector pGL-3basic. The mutation was performed within the predicted binding site GATTACGTATTT in the IFNG promoter. The primer sets used in mutation experiments were listed in Table 2. Subsequently, these recombinant plasmids were delivered into 293T cells in various combinations. The luciferase activity was measured with Dual-Luciferase Reporter Assay System (E1910) following the instruction of manufacturers.

### Statistical Analysis

Statistical analyses were performed by one-way ANOVA, two-way ANOVA or T-test using GraphPad Prism 5.0 software (Prism 5.0 GraphPad Software, San Diego, Calif.).

All patents, patent applications, and other publications, including GenBank Accession Numbers, cited in this application are incorporated by reference in the entirety for all purposes.

TABLE 1
Antibodies used in this study

| Target protein | Host | Conjugate | Company | Catalogue NO. |
|---|---|---|---|---|
| Hu-Smad3 | Rabbit | unconjugated | Abcam | Ab28379 |
| Hu-CD56 | Mouse | FITC | eBioscience | 11-0566-41 |
| Hu-E4BP4 | Rabbit | unconjugated | Cell Signaling | 14312 |
| Hu-β-actin | Mouse | unconjugated | Santa Cruz | Sc69879 |
| Hu-Smad3 | Rabbit | unconjugated | Cell Signaling | 9523s |
| (ChIP Assay) | | | | |
| Hu-E4BP4 | Rabbit | unconjugated | Cell Signaling | 14312 |
| (ChIP Assay) | | | | |

TABLE 2
Sequence of primers

| Gene | Forward Primers | Reverse Primers |
|---|---|---|
| Hu-Smad3 | CCCCAGAGCAATATTCCAGA | GACATCGGATTCGGGGATAG |
| Hu-IFN-γ | TGGTTGTCCTGCCTGCAATA | TAGGTTGGCTGCCTAGTTGG |
| Hu-Granzyme B | GCAGGAAGATCGAAAGTGCG | GGCATGCCATTGTTTCGTCC |
| Hu-Perforin | CTATACGGGATTCCAGCTCCA | ACCTTTGTGTGTCCACTGGG |
| Hu-NKG2D | CTGGGAGATGAGTGAATTTCA | GACTTCACCAGTTTAAGTAAA |
| | TA | TC |
| Hu-E4BP4 | ACATGTTGTCTGTTTGGTGTC | ATACAGCCTTCGCATGGACTA |
| | TTTTT | TC |
| Hu-GAPDH | GAAGGTGAAGGTCGGAGTC | GAAGATGGTGATGGGATTTC |
| Hu-NFIL3 (E4BP4)- | ATGCAATGGAGCAGGAGTTC | TGCACATGTTGTCTGTTTGG |
| 3′UTR | | |
| (ChIP Assay) | | |
| Hu-IFNG (IFN-γ)- | CTCCTCTGGCTGCTGGTATT | GTGGGCATAATGGGTCTGTC |
| promoter | | |
| (ChIP Assay) | | |
| E4bp4 3′UTR mutation | TTGTGTTATCACTCTGCCTGT | CAATATATACAGCCTTCGCATG |
| | GTATTCAGTCTATGTCCATGCG | GACATAGACTGAATACACAGG |
| | AAGGCTGTATATATTG | CAGAGTGATAACACAA |
| IFNG promoter mutation | ATCTCATCTTAAAAAACTTGT | ATACCTAATTGAAGTCTCCTG |
| | GAGGGCGTACGGCTCTCAGG | AGAGCCGTACGCCCTCACAA |
| | AGACTTCAATTAGGTAT | GTTTTTTAAGATGAGAT |

TABLE 3
Sequence of shRNA

| Target genes | ShRNA sequence (5′-3′) |
|---|---|
| hSmad3 | GGAGAAATGGTGCGAGAAG |
| hE4BP4 | TTAGATGTCATGTCAATAGGTGAGG |

### REFERENCES

1. Calaf G M, Zepeda A B, Castillo R L, Figueroa C A, Arias C, Figueroa E, et al. Molecular aspects of breast cancer resistance to drugs (Review). International journal of oncology 2015; 47(2):437-45.
2. Xu R, Wang Q. Large-scale automatic extraction of side effects associated with targeted anticancer drugs from full-text oncological articles. Journal of biomedical informatics 2015; 55:64-72.
3. Quail D F, Joyce J A. Microenvironmental regulation of tumor progression and metastasis. Nature medicine 2013; 19(11):1423-37.
4. Kantoff P W, Higano C S, Shore N D, Berger E R, Small E J, Penson D F, et al. Sipuleucel-T immunotherapy for castration-resistant prostate cancer. New England Journal of Medicine 2010; 363(5):411-22.
5. Pule M A, Savoldo B, Myers G D, Rossig C, Russell H V, Dotti G, et al. Virus-specific T cells engineered to coexpress tumor-specific receptors: persistence and antitumor activity in individuals with neuroblastoma. Nature medicine 2008; 14(11):1264-70.
6. Ruggeri L, Capanni M, Urbani E, Perruccio K, Shlomchik W D, Tosti A et al Effectiveness of donor natural killer cell alloreactivity in mismatched hematopoietic transplants. Science 2002; 295(5562):2097-100.
7. Stern M, Passweg J, Meyer-Monard S, Esser R, Tonn T, Soerensen J, et al. Pre-emptive immunotherapy with purified natural killer cells after haploidentical SCT: a prospective phase II study in two centers. Bone marrow transplantation 2013; 48(3):433-8.
8. Miller J S, Soignier Y, Panoskaltsis-Mortari A, McNearney S A, Yun G H, Fautsch S K, et al Successful adoptive transfer and in vivo expansion of human haploidentical NK cells in patients with cancer. Blood 2005; 105(8):3051-7.
9. Rubnitz J E, Inaba H, Ribeiro R C, Pounds S, Rooney B, Bell T, et al. NKAML: a pilot study to determine the safety and feasibility of haploidentical natural killer cell transplantation in childhood acute myeloid leukemia. Journal of clinical oncology 2010; 28(6):955-9.
10. Curti A, Ruggeri L, D'Addio A, Bontadini A, Dan E, Motta M R, et al. Successful transfer of alloreactive haploidentical KIR ligand-mismatched natural killer cells after infusion in elderly high risk acute myeloid leukemia patients. Blood 2011; 118(12):3273-9.
11. Bachanova V, Cooley S, Defor T E, Verneris M R, Zhang B, McKenna D H, et al Clearance of acute myeloid leukemia by haploidentical natural killer cells is improved using IL-2 diphtheria toxin fusion protein. Blood 2014; 123(25):3855-63.
12. Rusakiewicz S, Semeraro M, Sarabi M, Desbois M, Locher C, Mendez R, et al Immune infiltrates are prognostic factors in localized gastrointestinal stromal tumors. Cancer research 2013; 73(12):3499-510.
13. Mamessier E, Sylvain A, Thibult M-L, Houvenaeghel G, Jacquemier J, Castellano R, et al. Human breast cancer cells enhance self tolerance by promoting evasion from NK cell antitumor immunity. The Journal of clinical investigation 2011; 121(9):3609-22.
14. Stringaris K, Sekine T, Khoder A, Alsuliman A, Razzaghi B, Sargeant R, et al Leukemia-induced phenotypic and functional defects in natural killer cells predict failure to achieve remission in acute myeloid leukemia. Haematologica 2014; 99(5):836-47.
15. Rouce R H, Shaim H, Sekine T, Weber G, Ballard B, Ku S, et al. The TGF-β/SMAD pathway is an important mechanism for NK cell immune evasion in childhood B-acute lymphoblastic leukemia. Leukemia 2016; 30(4):800-11.
16. Li M O, Wan Y Y, Sanjabi S, Robertson A-K L, Flavell R A. Transforming growth factor-β regulation of immune responses. Annu Rev Immunol 2006; 24:99-146.
17. Rook A H, Kehrl J H, Wakefield L M, Roberts A B, Sporn M B, Burlington D B, et al Effects of transforming growth factor beta on the functions of natural killer cells: depressed cytolytic activity and blunting of interferon responsiveness. The Journal of Immunology 1986; 136(10):3916-20.
18. Bellone G, Aste-Amezaga M, Trinchieri G, Rodeck U. Regulation of NK cell functions by TGF-beta 1. The Journal of Immunology 1995; 155(3):1066-73.
19. Trotta R, Dal Col J, Yu J, Ciarlariello D, Thomas B, Zhang X, et al. TGF-β utilizes SMAD3 to inhibit CD16-mediated IFN-γ production and antibody-dependent cellular cytotoxicity in human NK cells. The Journal of Immunology 2008; 181(6):3784-92.
20. Nam J-S, Terabe M, Mamura M, Kang M-J, Chae H, Stuelten C, et al. An anti-transforming growth factor β antibody suppresses metastasis via cooperative effects on multiple cell compartments. Cancer research 2008; 68(10):3835-43.
21. Schlingensiepen K H, Jaschinski F, Lang S A, Moser C, Geissler E K, Schlitt H J, et al Transforming growth factor-beta 2 gene silencing with trabedersen (A P 12009) in pancreatic cancer. Cancer science 2011; 102(6):1193-200.
22. Gordon M S, Ilaria R, de Alwis D P, Mendelson D S, McKane S, Wagner M M, et al. A phase I study of tasisulam sodium (LY573636 sodium), a novel anticancer compound, administered as a 24-h continuous infusion in patients with advanced solid tumors. Cancer chemotherapy and pharmacology 2013; 71(1):21-7.
23. Park C-Y, Son J-Y, Jin C H, Nam J-S, Kim D-K, Sheen Y Y. EW-7195, a novel inhibitor of ALK5 kinase inhibits EMT and breast cancer metastasis to lung. European journal of cancer 2011; 47(17):2642-53.
24. Zhong Z, Carroll K D, Policarpio D, Osborn C, Gregory M, Bassi R, et al. Anti-Transforming Growth Factor β Receptor II Antibody Has Therapeutic Efficacy against Primary Tumor Growth and Metastasis through Multieffects on Cancer, Stroma, and Immune Cells. Clinical Cancer Research 2010; 16(4):1191-205.
25. Subramaniam V, Ace O, Prud'homme G J, Jothy S. Tranilast treatment decreases cell growth, migration and inhibits colony formation of human breast cancer cells. Experimental and molecular pathology 2011; 90(1):116-22.
26. Kocic J, Bugarski D, Santibanez J F. SMAD3 is essential for transforming growth factor-β1-induced urokinase type plasminogen activator expression and migration in transformed keratinocytes. European journal of cancer 2012; 48(10):1550-7.
27. Shull M M, Ormsby I, Kier A B, Pawlowskr S, Diebold R J, Yin M, et al. Targeted disruption of the mouse transforming growth factor-β1 gene results in multifocal inflammatory disease. Nature 1992; 359(6397):693.
28. Millet C, Zhang Y E. Roles of Smad3 in TGF-β signaling during carcinogenesis. Critical Reviews' in Eukaryotic Gene Expression 2007; 17(4).
29. Tang P, Zhou S, Meng X-M, Wang Q-M, Li C-J, Lian G-Y, et al. Smad3 Promotes Cancer Progression by Inhibiting E4BP4-mediated NK Cell Development. Nature Communications 2017; In Press.
30. Jinnin M, Ihn H, Tamaki K. Characterization of SIS3, a novel specific inhibitor of Smad3, and its effect on transforming growth factor-β1-induced extracellular matrix expression. Molecular pharmacology 2006; 69(2):597-607.
31. Gascoyne D M, Long E, Veiga-Fernandes H, De Boer J, Williams O, Seddon B, et al The basic leucine zipper transcription factor E4BP4 is essential for natural killer cell development. Nature immunology 2009; 10(10):1118-24.
32. Subramanian A, Tamayo P, Mootha V K, Mukherjee S, Ebert B L, Gillette M A et al Gene set enrichment analysis: a knowledge-based approach for interpreting genome-wide expression profiles. Proceedings of the National Academy of Sciences 2005; 102(43):15545-50.
33. Ruggeri L, Capanni M, Urbani E, Perruccio K, Shlomchik W D, Tosti A et al Effectiveness of donor natural killer cell alloreactivity in mismatched hematopoietic transplants. Science 2002; 295(5562):2097-100 doi 10.1126/science.1068440.
34. Sakamoto N, Ishikawa T, Kokura S, Okayama T, Oka K, Ideno M, et al. Phase I clinical trial of autologous NK cell therapy using novel expansion method in patients with advanced digestive cancer. Journal of translational medicine 2015; 13(1):1.
35. Parkhurst M R, Yang J C, Langan R C, Dudley M E, Nathan D-A N, Feldman S A, et al. T cells targeting carcinoembryonic antigen can mediate regression of metastatic colorectal cancer but induce severe transient colitis. Molecular Therapy 2011; 19(3):620-6.
36. Shi J, Tricot G, Szmania S, Rosen N, Garg T K, Malaviarachchi P A, et al. Infusion of haplo—identical killer immunoglobulin—like receptor ligand mismatched NK cells for relapsed myeloma in the setting of autologous stem cell transplantation. British journal of haematology 2008; 143(5):641-53.
37. Bachanova V, Burns L J, McKenna D H, Curtsinger J, Panoskaltsis-Mortari A, Lindgren B R, et al. Allogeneic natural killer cells for refractory lymphoma. Cancer Immunol Immunother 2010; 59(11):1739-44 doi 10.1007/s00262-010-0896-z.
38. Nagashima S, Mailliard R, Kashii Y, Reichert T E, Herberman R B, Robbins P, et al Stable transduction of the interleukin-2 gene into human natural killer cell lines and their phenotypic and functional characterization in vitro and in vivo. Blood 1998; 91(10):3850-61.
39. Imamura M, Shook D, Kamiya T, Shimasaki N, Chai S M, Coustan-Smith E, et al Autonomous growth and increased cytotoxicity of natural killer cells expressing membrane-bound interleukin-15. Blood 2014; 124(7):1081-8.
40. Chang Y-H, Connolly J, Shimasaki N, Mimura K, Kono K, Campana D. A chimeric receptor with NKG2D specificity enhances natural killer cell activation and killing of tumor cells. Cancer research 2013; 73(6):1777-86.
41. Figueiredo C, Seltsam A, Blasczyk R. Permanent silencing of NKG2A expression for cell-based therapeutics. Journal of molecular medicine 2009; 87(2):199-210.
42. Binyamin L, Alpaugh R K, Hughes T L, Lutz C T, Campbell K S, Weiner L M. Blocking NK cell inhibitory self-recognition promotes antibody-dependent cellular cytotoxicity in a model of anti-lymphoma therapy. The Journal of Immunology 2008; 180(9):6392-401.
43. Somanchi S S, Somanchi A, Cooper L J, Lee D A. Engineering lymph node homing of ex vivo-expanded human natural killer cells via trogocytosis of the chemokine receptor CCR7. Blood 2012; 119(22):5164-72.
44. Boissel L, Betancur M, Wels W S, Tuncer H, Klingemann H. Transfection with mRNA for CD19 specific chimeric antigen receptor restores NK cell mediated killing of CLL cells. Leukemia research 2009; 33(9):1255-9.
45. Müller T, Uherek C, Maki G, Chow K U, Schimpf A, Klingemann H-G, et al Expression of a CD20-specific chimeric antigen receptor enhances cytotoxic activity of NK cells and overcomes NK-resistance of lymphoma and leukemia cells. Cancer Immunology, Immunotherapy 2008; 57(3):411-23.
46. Kruschinski A, Moosmann A, Poschke I, Norell H, Chmielewski M, Seliger B, et al Engineering antigen-specific primary human NK cells against HER-2 positive carcinomas. Proceedings of the National Academy of Sciences 2008; 105(45):17481-6.
47. Liu H, Yang B, Sun T, Lin L, Hu Y, Deng M, et al. Specific growth inhibition of ErbB2-expressing human breast cancer cells by genetically modified NK-92 cells. Oncology reports 2015; 33(1):95-102.
48. Schirrmann T, Pecher G. Human natural killer cell line modified with a chimeric immunoglobulin T-cell receptor gene leads to tumor growth inhibition in vivo. Cancer gene therapy 2002; 9(4):390-8.
49. Zhang G, Liu R, Zhu X, Wang L, Ma J, Han H, et al. Retargeting NK-92 for anti-melanoma activity by a TCR-like single-domain antibody. Immunology and cell biology 2013; 91(10):615-24.
50. Sahm C, Schönfeld K, Wels W S. Expression of IL-15 in NK cells results in rapid enrichment and selective cytotoxicity of gene-modified effectors that carry a tumor-specific antigen receptor. Cancer Immunology, Immunotherapy 2012; 61(9):1451-61.
51. Wilson E B, El-Jawhari J J, Neilson A L, Hall G D, Melcher A A, Meade J L, et al. Human tumour immune evasion via TGF-β blocks NK cell activation but not survival allowing therapeutic restoration of anti-tumour activity. PloS one 2011; 6(9):e22842.
52. Meadows S K, Eriksson M, Barber A, Sentman C L. Human NK cell IFN-γ production is regulated by endogenous TGF-β. International immunopharmacology 2006; 6(6):1020-8.
53. Castriconi R, Cantoni C, Della Chiesa M, Vitale M, Marcenaro E, Conte R, et al Transforming growth factor 131 inhibits expression of NKp30 and NKG2D receptors: consequences for the NK-mediated killing of dendritic cells. Proceedings of the National Academy of Sciences 2003; 100(7):4120-5.
54. Yu J, Wei M, Becknell B, Trotta R, Liu S, Boyd Z, et al. Pro- and antiinflammatory cytokine signaling: reciprocal antagonism regulates interferon-gamma production by human natural killer cells. Immunity 2006; 24(5):575-90.
55. Thomas D A, Massagué J. TGF-β directly targets cytotoxic T cell functions during tumor evasion of immune surveillance. Cancer cell 2005; 8(5):369-80.
56. Male V, Nisoli I, Gascoyne D M, Brady H J. E4BP4: an unexpected player in the immune response. Trends in immunology 2012; 33(2):98-102.
57. Yang B, Liu H, Shi W, Wang Z, Sun S, Zhang G, et al. Blocking transforming growth factor-β signaling pathway augments antitumor effect of adoptive NK-92 cell therapy. International immunopharmacology 2013; 17(2):198-204.
58. Yang X, Letterio J J, Lechleider R J, Chen L, Hayman R, Gu H, et al. Targeted disruption of SMAD3 results in impaired mucosal immunity and diminished T cell responsiveness to TGF-β. The EMBO journal 1999; 18(5): 1280-91.
59. Pittari G, Filippini P, Gentilcore G, Grivel J C, Rutella S. Revving up Natural Killer Cells and Cytokine-Induced Killer Cells Against Hematological Malignancies. Front Immunol 2015; 6:230 doi 10.3389/fimmu.2015.00230.
60. Tonn T, Becker S, Esser R, Schwabe D, Seifried E. Cellular immunotherapy of malignancies using the clonal natural killer cell line NK-92. Journal of hematotherapy & stem cell research 2001; 10(4):535-44.
61. Arai S, Meagher R, Swearingen M, Myint H, Rich E, Martinson J, et al. Infusion of the allogeneic cell line NK-92 in patients with advanced renal cell cancer or melanoma: a phase I trial. Cytotherapy 2008; 10(6):625-32.
62. Sharpe M, Mount N. Genetically modified T cells in cancer therapy: opportunities and challenges. Disease Models and Mechanisms 2015; 8(4):337-50.
63. Kershaw M H, Westwood J A, Slaney C Y, Darcy P K. Clinical application of genetically modified T cells in cancer therapy. Clinical & translational immunology 2014; 3(5):e16.
64. Carlsten M, Childs R W. Genetic manipulation of NK cells for cancer immunotherapy: techniques and clinical implications. Frontiers in immunology 2015; 6.
65. Micucci F, Zingoni A, Piccoli M, Frati L, Santoni A, Galandrini R. High-efficient lentiviral vector-mediated gene transfer into primary human NK cells. Experimental hematology 2006; 34(10):1344-52.
66. McGarrity G J, Hoyah G, Winemiller A, Andre K, Stein D, Blick G, et al. Patient monitoring and follow-up in lentiviral clinical trials. The journal of gene medicine 2013; 15(2):78-82.
67. MacMicking J, Xie Q-w, Nathan C. Nitric oxide and macrophage function. Annual review of immunology 1997; 15(1):323-50.
68. Ikeda H, Old L J, Schreiber R D. The roles of IFNγ in protection against tumor development and cancer immunoediting. Cytokine & growth factor reviews 2002; 13(2):95-109.
69. Shultz L D, Schweitzer P A, Christianson S W, Gott B, Schweitzer I B, Tennent B, et al Multiple defects in innate and adaptive immunologic function in NOD/LtSz-scid mice. The Journal of Immunology 1995; 154(4180-91.
70. Meyerrose T E, Herrbrich P, Hess D A, Nolta J A. Immune-deficient mouse models for analysis of human stem cells. 2003.

What is claimed is:

1. A modified natural killer (NK) cell, wherein Smad3 activity in the modified NK cell is inhibited compared to a unmodified parent NK cell.

2. The modified NK cell of claim 1, wherein Smad3 activity is inhibited by 50%, 70%, 80% or more compared to the unmodified parent NK cell.

3. The modified NK cell of claim 1, wherein Smad3 activity is abolished.

4. The modified NK cell of claim 1, wherein the Smad3 genomic sequence has been altered.

5. The modified NK cell of claim 1, comprising in its genome an exogenous sequence encoding a polynucleotide sequence that corresponds to or is complementary to at least a segment of the Smad3 genomic sequence.

6. The modified NK cell of claim 1, wherein the parent NK cell is human NK92 cell.

7. A composition comprising the modified NK cell of claim 1 and a physiologically acceptable excipient.

8. The composition of claim 7, which is formulated for injection.

9. The composition of claim 8, which is formulated for subcutaneous, intramuscular, intravenous, intraperitoneal, or intratumoral injection.

10. The composition of claim 9, which is formulated in a dosage form for administration to a patient.

11. A method for treating cancer, comprising administration to a cancer patient an effective number of the modified NK cell of claim 1.

12. The method of claim 11, wherein the administration comprises injection.

13. The method of claim 12, wherein the injection is subcutaneous, intramuscular, intravenous, intraperitoneal, or intratumoral injection.

14. The method of claim 11, wherein the administration is performed daily, once every two days, weekly, once every two weeks, or monthly.

15. The method of claim 11, further comprising administration of a second anti-cancer therapeutic agent to the patient.


SEQUENCE LISTING (35 sequences)

SEQ ID NO: 1 - DNA, 20 bp, Artificial Sequence
  synthetic Hu-Smad3 forward primer
ccccagagcaatattccaga

SEQ ID NO: 2 - DNA, 20 bp, Artificial Sequence
  synthetic Hu-IFN-gamma forward primer
tggttgtcctgcctgcaata

SEQ ID NO: 3 - DNA, 20 bp, Artificial Sequence
  synthetic Hu-Granzyme B forward primer
gcaggaagatcgaaagtgcg

SEQ ID NO: 4 - DNA, 21 bp, Artificial Sequence
  synthetic Hu-Perforin forward primer
ctatacgggattccagctcca

SEQ ID NO: 5 - DNA, 23 bp, Artificial Sequence
  synthetic Hu-NKG2D forward primer
ctgggagatgagtgaatttcata

SEQ ID NO: 6 - DNA, 26 bp, Artificial Sequence
  synthetic Hu-E4BP4 forward primer
acatgttgtctgtttggtgtcttttt

SEQ ID NO: 7 - DNA, 19 bp, Artificial Sequence
  synthetic Hu-GAPDH forward primer
gaaggtgaaggtcggagtc

SEQ ID NO: 8 - DNA, 20 bp, Artificial Sequence
  synthetic Hu-NFIL3 (E4BP4)-3UTR (ChIP Assay) forward primer
atgcaatggagcaggagttc

SEQ ID NO: 9 - DNA, 20 bp, Artificial Sequence
  synthetic Hu-IFNG (IFN-gamma)-promoter (ChIP Assay) forward primer
ctcctctggctgctggtatt

SEQ ID NO: 10 - DNA, 59 bp, Artificial Sequence
  synthetic E4bp4 3UTR mutation forward primer
ttgtgttatcactctgcctgtgtattcagtctatgtccatgcgaaggctgtatatattg

SEQ ID NO: 11 - DNA, 58 bp, Artificial Sequence
  synthetic IFNG promoter mutation forward primer
atctcatcttaaaaaacttgtgagggcgtacggctctcaggagacttcaattaggtat

SEQ ID NO: 12 - DNA, 20 bp, Artificial Sequence
  synthetic Hu-Smad3 reverse primer
gacatcggattcggggatag

SEQ ID NO: 13 - DNA, 20 bp, Artificial Sequence
  synthetic Hu-IFN-gamma reverse primer
taggttggctgcctagttgg

SEQ ID NO: 14 - DNA, 20 bp, Artificial Sequence
  synthetic Hu-Granzyme B reverse primer
ggcatgccattgtttcgtcc

SEQ ID NO: 15 - DNA, 20 bp, Artificial Sequence
  synthetic Hu-Perforin reverse primer
acctttgtgtgtccactggg

SEQ ID NO: 16 - DNA, 23 bp, Artificial Sequence
  synthetic Hu-NKG2D reverse primer
gacttcaccagtttaagtaaatc

SEQ ID NO: 17 - DNA, 23 bp, Artificial Sequence
  synthetic Hu-E4BP4 reverse primer
atacagccttcgcatggactatc

SEQ ID NO: 18 - DNA, 20 bp, Artificial Sequence
  synthetic Hu-GAPDH reverse primer
gaagatggtgatgggatttc

SEQ ID NO: 19 - DNA, 20 bp, Artificial Sequence
  synthetic Hu-NFIL3 (E4BP4)-3UTR (ChIP Assay) reverse primer
tgcacatgttgtctgtttgg

SEQ ID NO: 20 - DNA, 20 bp, Artificial Sequence
  synthetic Hu-IFNG (IFN- gamma)-promoter (ChIP Assay) reverse primer
gtgggcataatgggtctgtc

SEQ ID NO: 21 - DNA, 59 bp, Artificial Sequence
  synthetic E4bp4 3UTR mutation reverse primer
caatatatacagccttcgcatggacatagactgaatacacaggcagagtgataacacaa

SEQ ID NO: 22 - DNA, 58 bp, Artificial Sequence
  synthetic IFNG promoter mutation reverse primer
atacctaattgaagtctcctgagagccgtacgccctcacaagttttttaagatgagat

SEQ ID NO: 23 - DNA, 19 bp, Artificial Sequence
  synthetic ShRNA target sequence for hSmad3 gene
ggagaaatggtgcgagaag

SEQ ID NO: 24 - DNA, 25 bp, Artificial Sequence
  synthetic ShRNA target sequence for hE4BP4 gene
ttagatgtcatgtcaataggtgagg

SEQ ID NO: 25 - DNA, 12 bp, Artificial Sequence
  synthetic predicted binding site sequence in IFNG promoter
gattacgtattt

SEQ ID NO: 26 - DNA, 20 bp, Homo sapiens
atggactatctgactataca

SEQ ID NO: 27 - DNA, 19 bp, Mus musculus
gggggctatctactacacg

SEQ ID NO: 28 - DNA, 24 bp, Homo sapiens
cctgaggattacgtattttcacaa

SEQ ID NO: 29 - DNA, 24 bp, Mus musculus
cctcgggattacgtattttcacaa

SEQ ID NO: 30 - DNA, 80 bp, Artificial Sequence
  synthetic consensus sequence to target human SMAD3 mRNA
  misc_feature (74)..(80): n is a, c, g, or t
aggacgaggatccggagaaatggtgcgagaagttcaagagacttctcgcaccatttctccttttttctcgaggnnnnnnn

SEQ ID NO: 31 - DNA, 62 bp, Artificial Sequence
  synthetic ShRNA-1-hSmad3 sequence
ccggagaaatggtgcgagaagttcaagagacttctcgcaccatttctccttttttctcgagg

SEQ ID NO: 32 - DNA, 83 bp, Artificial Sequence
  synthetic 2373540.2.bw2014 sequence
aggacgaggatccggagaaatggtgcgagaagttcaagagacttctcgcaccatttctccttttttctcgaggaggaattcta

SEQ ID NO: 33 - DNA, 120 bp, Artificial Sequence
  synthetic consensus sequence to target human E4BP4 mRNA
  misc_feature (104)..(120): n is a, c, g, or t
ttcttggctttatatatcttgtggaaaggacgaggatccttagatgtcatgtcaataggtgaggtcaagagacctcacctattgacatgacatctaattttttnnnnnnnnnnnnnnnnn

SEQ ID NO: 34 - DNA, 70 bp, Artificial Sequence
  synthetic 111.seq sequence
gatccttagatgtcatgtcaataggtgaggttcaagagacctcacctattgacatgacatctaatttttt

SEQ ID NO: 35 - DNA, 120 bp, Artificial Sequence
  synthetic 2925732.1.BW2015 sequence
ttcttggctttatatatcttgtggaaaggacgaggatccttagatgtcatgtcaataggtgaggtcaagagacctcacctattgacatgacatctaattttttacgcgtctcgagttctg